SEQUENCE LISTING (75 sequences)

SEQ ID NO: 1 - protein, 129 aa, Homo sapiens
QNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIR

SEQ ID NO: 2 - protein, 124 aa, Homo sapiens [sequence omitted]

SEQ ID NO: 3 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 4 - protein, 122 aa, Homo sapiens
QVQLQESGGGLVQAGGSLRLSCAASGNISASYEMGWYRQAPGKERELVAAIDSGASTYYADSVKGRFTISRDNAKNTVYLQMNSLKPEDTAVYYCAAAGPSYGAPDVIHFYWGQGTQVTVSS

SEQ ID NO: 5 - protein, 9 aa, Homo sapiens
GNISASYEM

SEQ ID NO: 6 - protein, 14 aa, Homo sapiens
ELVAAIDSGASTYY

SEQ ID NO: 7 - protein, 16 aa, Homo sapiens
AAAGPSYGAPDVIHFY

SEQ ID NO: 8 - protein, 118 aa, Homo sapiens
QVQLQESGGGLVQAGGSLRLSCAASGTISGRQAMGWYRQAPGKEREFVAGIAYGATTNYADSVKGRFTISRDNAKNTVYLQMNSLKPEDTAVYYCAAVAVDPEVLWYWGQGTQVTVSS

SEQ ID NO: 9 - protein, 9 aa, Homo sapiens
GTISGRQAM

SEQ ID NO: 10 - protein, 14 aa, Homo Sapiens
EFVAGIAYGATTNY

SEQ ID NO: 11 - protein, 12 aa, Homo sapiens
AAVAVDPEVLWY

SEQ ID NO: 12 - protein, 118 aa, Homo sapiens
QVQLQESGGGLVQAGGSLRLSCAASGTIFYFSIMGWYRQAPGKEREFVAGIGVGSNTYYADSVKGRFTISRDNAKNTVYLQMNSLKPEDTAVYYCAAFDSISGELQYWGQGTQVTVSS

SEQ ID NO: 13 - protein, 9 aa, Homo sapiens
GTIFYFSIM

SEQ ID NO: 14 - protein, 14 aa, Homo sapiens
EFVAGIGVGSNTYY

SEQ ID NO: 15 - protein, 12 aa, Homo sapiens
AAFDSISGELQY

SEQ ID NO: 16 - protein, 118 aa, Homo sapiens
QVQLQESGGGLVQAGGSLRLSCAASGSIFYRGVMGWYRQAPGKERELVAAINDGATTYYADSVKGRFTISRDNAKNTVYLQMNSLKPEDTAVYYCAVWHRRTGVLLYWGQGTQVTVSS

SEQ ID NO: 17 - protein, 9 aa, Homo sapiens
GSIFYRGVM

SEQ ID NO: 18 - protein, 14 aa, Homo sapiens
ELVAAINDGATTYY

SEQ ID NO: 19 - protein, 12 aa, Homo sapiens
AVWHRRTGVLLY

SEQ ID NO: 20 - protein, 118 aa, Homo sapiens
QVQLQESGGGLVQAGGSLRLSCAASGTISVRDPMGWYRQAPGKEREFVAGIGQGSTTNYADSVKGRFTISRDNAKNTVYLQMNSLKPEDTAVYYCAVLYWRQDWHLYWGQGTQVTVSS

SEQ ID NO: 21 - protein, 9 aa, Homo sapiens
GTISVRDPM

SEQ ID NO: 22 - protein, 14 aa, Homo sapiens
EFVAGIGQGSTTNY

SEQ ID NO: 23 - protein, 12 aa, Homo sapiens
AVLYWRQDWHLY

SEQ ID NO: 24 - protein, 118 aa, Homo sapiens
QVQLQESGGGLVQAGGSLRLSCAASGSIFGGDAMGWYRQAPGKERELVAGINAGTSTYYADSVKGRFTISRDNAKNTVYLQMNSLKPEDTAVYYCAVAGYFPYVHTYWGQGTQVTVSS

SEQ ID NO: 25 - protein, 9 aa, Homo sapiens
GSIFGGDAM

SEQ ID NO: 26 - protein, 14 aa, Homo sapiens
ELVAGINAGTSTYY

SEQ ID NO: 27 - protein, 12 aa, Homo sapiens
AVAGYFPYVHTY

SEQ ID NO: 28 - protein, 122 aa, Homo sapiens
QVQLQESGGGLVQAGGSLRLSCAASGTISDGPVMGWYRQAPGKEREFVAGIGYGTNTNYADSVKGRFTISRDNAKNTVYLQMNSLKPEDTAVYYCAVIVPYNSDRSLYLWYWGQGTQVTVSS

SEQ ID NO: 29 - protein, 9 aa, Homo sapiens
GTISDGPVM

SEQ ID NO: 30 - protein, 14 aa, Homo sapiens
EFVAGIGYGTNTNY

SEQ ID NO: 31 - protein, 16 aa, Homo sapiens
AVIVPYNSDRSLYLWY

SEQ ID NO: 32 - protein, 127 aa, Homo sapiens
QVQLQESGGGLVQAGGSLRLSCAASGNISYYRHMGWYRQAPGKERELVASIGDGGNTNYADSVKGRFTISRDNAKNTVYLQMNSLKPEDTAVYYCAVVHNTYLLYDPYVWDYLLLYWGQGTQVTVSS

SEQ ID NO: 33 - protein, 9 aa, Homo sapiens
GTISRQQTM

SEQ ID NO: 34 - protein, 14 aa, Homo sapiens
EFVASIAQGGNTYY

SEQ ID NO: 35 - protein, 14 aa, Homo sapiens
AVYERSDTEYYLRY

SEQ ID NO: 36 - protein, 122 aa, Homo sapiens
QVQLQESGGGLVQAGGSLRLSCAASGTIFWTASMGWYRQAPGKERELVAGITAGATTYYADSVKGRFTISRDNAKNTVYLQMNSLKPEDTAVYYCAARTPDRTGIAKWHDYWGQGTQVTVSS

SEQ ID NO: 37 - protein, 9 aa, Homo sapiens
GTIFWTASM

SEQ ID NO: 38 - protein, 14 aa, Homo sapiens
ELVAGITAGATTYY

SEQ ID NO: 39 - protein, 16 aa, Homo sapiens
AARTPDRTGIAKWHDY

SEQ ID NO: 40 - protein, 118 aa, Homo sapiens
QVQLQESGGGLVQAGGSLRLSCAASGTIFIPLGMGWYRQAPGKERELVAGITYGGITYYADSVKGRFTISRDNAKNTVYLQMNSLKPEDTAVYYCAARYVVYETHDYWGQGTQVTVSS

SEQ ID NO: 41 - protein, 9 aa, Homo sapiens
GTIFIPLGM

SEQ ID NO: 42 - protein, 14 aa, Homo sapiens
ELVAGITYGGITYY

SEQ ID NO: 43 - protein, 12 aa, Homo sapiens
AARYVVYETHDY

SEQ ID NO: 44 - protein, 117 aa, Homo sapiens
QVQLQESGGGLVQAGGSLRLSCAASGTISPRIMGWYRQAPGKEREFVASINDGASTNYADSVKGRFTISRDNAKNTVYLQMNSLKPEDTAVYYCAAAVWYDGWLVYWGQGTQVTVSS

SEQ ID NO: 45 - protein, 8 aa, Homo sapiens
GTISPRIM

SEQ ID NO: 46 - protein, 14 aa, Homo sapiens
EFVASINDGASTNY

SEQ ID NO: 47 - protein, 12 aa, Homo sapiens
AAAVWYDGWLVY

SEQ ID NO: 48 - protein, 122 aa, Homo sapiens
QVQLQESGGGLVQAGGSLRLSCAASGNIFRAVNMGWYRQAPGKERELVAAITTGANTYYADSVKGRFTISRDNAKNTVYLQMNSLKPEDTAVYYCAARWYPAPGFNHYYDYWGQGTQVTVSS

SEQ ID NO: 49 - protein, 9 aa, Homo sapiens
GNIFRAVNM

SEQ ID NO: 50 - protein, 14 aa, Homo sapiens
ELVAAITTGANTYY

SEQ ID NO: 51 - protein, 16 aa, Homo sapiens
AARWYPAPGFNHYYDY

SEQ ID NO: 52 - protein, 122 aa, Homo sapiens
QVQLQESGGGLVQAGGSLRLSCAASGSISSATVMGWYRQAPGKEREFVAAITAGGTTNYADSVKGRFTISRDNAKNTVYLQMNSLKPEDTAVYYCAVVVFTSQDRIYYYRYWGQGTQVTVSS

SEQ ID NO: 53 - protein, 9 aa, Homo sapiens
GSISSATVM

SEQ ID NO: 54 - protein, 14 aa, Homo sapiens
EFVAAITAGGTTNY

SEQ ID NO: 55 - protein, 16 aa, Homo sapiens
AVVVFTSQDRIYYYRY

SEQ ID NO: 56 - protein, 122 aa, Homo sapiens
QVQLQESGGGLVQAGGSLRLSCAASGSIFPAQVMGWYRQAPGKERELVAGINYGGSTYYADSVKGRFTISRDNAKNTVYLQMNSLKPEDTAVYYCAVFQWDDNDGRYYLGYWGQGTQVTVSS

SEQ ID NO: 57 - protein, 9 aa, Homo sapiens
GSIFPAQVM

SEQ ID NO: 58 - protein, 14 aa, Homo sapiens
ELVAGINYGGSTYY

SEQ ID NO: 59 - protein, 16 aa, Homo sapiens
AVFQWDDNDGRYYLGY

SEQ ID NO: 60 - protein, 122 aa, Homo sapiens
QVQLQESGGGLVQAGGSLRLSCAASGTIFHGSTMGWYRQAPGKERELVAGIASGSSTYYADSVKGRFTISRDNAKNTVYLQMNSLKPEDTAVYYCAVAQQQHGTDRSWFIYWGQGTQVTVSS

SEQ ID NO: 61 - protein, 9 aa, Homo sapiens
GTIFHGSTM

SEQ ID NO: 62 - protein, 14 aa, Homo sapiens
ELVAGIASGSSTYY

SEQ ID NO: 63 - protein, 16 aa, Homo sapiens
AVAQQQHGTDRSWFIY

SEQ ID NO: 64 - protein, 122 aa, Homo sapiens
QVQLQESGGGLVQAGGSLRLSCAASGTISIYGPMGWYRQAPGKERELVAGISLGGSTNYADSVKGRFTISRDNAKNTVYLQMNSLKPEDTAVYYCAAASIAKSDYPYWHLYWGQGTQVTVSS

SEQ ID NO: 65 - protein, 9 aa, Homo sapiens
GTISIYGPM

SEQ ID NO: 66 - protein, 14 aa, Homo sapiens
ELVAGISLGGSTNY

SEQ ID NO: 67 - protein, 16 aa, Homo sapiens
AAASIAKSDYPYWHLY

SEQ ID NO: 68 - protein, 126 aa, Homo sapiens
QVQLQESGGGLVQAGGSLRLSCAASGTISARKRMGWYRQAPGKEREFVATIDYGTTTNYADSVKGRFTISRDNAKNTVYLQMNSLKPEDTAVYYCAVIDYVPIDYYQYAAGWHFYWGQGTQVTVSS

SEQ ID NO: 69 - protein, 9 aa, Homo sapiens
GNISPTYLM

SEQ ID NO: 70 - protein, 14 aa, Homo sapiens
EFVAGIAHGASTNY

SEQ ID NO: 71 - protein, 12 aa, Homo sapiens
AVNPYALDVLVY

SEQ ID NO: 72 - protein, 252 aa, Artificial sequence
  Synthetic sequence
QVQLQESGGGLVQAGGSLRLSCAASGNISASYEMGWYRQAPGKERELVAAIDSGASTYYADSVKGRFTISRDNAKNTVYLQMNSLKPEDTAVYYCAAAGPSYGAPDVIHFYWGQGTQVTVSSGGGGSGGGQVQLQESGGGLVQAGGSLRLSCAASGTISIYGPMGWYRQAPGKERELVAGISLGGSTNYADSVKGRFTISRDNAKNTVYLQMNSLKPEDTAVYYCAAASIAKSDYPYWHLYWGQGTQVTVSS

SEQ ID NO: 73 - protein, 248 aa, Artificial sequence
  Synthetic sequence
QVQLQESGGGLVQAGGSLRLSCAASGTIFYFSIMGWYRQAPGKEREFVAGIGVGSNTYYADSVKGRFTISRDNAKNTVYLQMNSLKPEDTAVYYCAAFDSISGELQYWGQGTQVTVSSGGGGSGGGQVQLQESGGGLVQAGGSLRLSCAASGTISIYGPMGWYRQAPGKERELVAGISLGGSTNYADSVKGRFTISRDNAKNTVYLQMNSLKPEDTAVYYCAAASIAKSDYPYWHLYWGQGTQVTVSS

SEQ ID NO: 74 - protein, 248 aa, Artificial sequence
  Synthetic sequence
QVQLQESGGGLVQAGGSLRLSCAASGTIFYFSIMGWYRQAPGKEREFVAGIGVGSNTYYADSVKGRFTISRDNAKNTVYLQMNSLKPEDTAVYYCAAFDSISGELQYWGQGTQVTVSSGGGGSGGGQVQLQESGGGLVQAGGSLRLSCAASGSISSATVMGWYRQAPGKEREFVAAITAGGTTNYADSVKGRFTISRDNAKNTVYLQMNSLKPEDTAVYYCAVVVFTSQDRIYYYRYWGQGTQVTVSS

SEQ ID NO: 75 - protein, 248 aa, Artificial sequence
  Synthetic sequence
QVQLQESGGGLVQAGGSLRLSCAASGSIFYRGVMGWYRQAPGKERELVAAINDGATTYYADSVKGRFTISRDNAKNTVYLQMNSLKPEDTAVYYCAVWHRRTGVLLYWGQGTQVTVSSGGGGSGGGQVQLQESGGGLVQAGGSLRLSCAASGSISSATVMGWYRQAPGKEREFVAAITAGGTTNYADSVKGRFTISRDNAKNTVYLQMNSLKPEDTAVYYCAVVVFTSQDRIYYYRYWGQGTQVTVSS

(19) United States
(12) Patent Application Publication
(10) Pub. No.: US 2024/0270854 A1
(43) Pub. Date: Aug. 15, 2024

(54) ENGINEERED BMP2 SURROGATE PROTEINS

(71) Applicant: The Board of Trustees of the Leland Stanford Junior University, Stanford, CA (US)

(72) Inventors: Charles K.F. Chan, Redwood City, CA (US); Liming Zhao, Redwood City, CA (US); Sicong Wang, Stanford, CA (US); Michael T. Longaker, Redwood City, CA (US); Eri Takematsu, Redwood City, CA (US)

(21) Appl. No.: 18/565,026

(22) PCT Filed: May 24, 2022
(86) PCT No.: PCT/US2022/030708

§ 371 Date: Nov. 28, 2023

Related U.S. Application Data
(60) Provisional application No. 63/196,540, filed on Jun. 3, 2021.

(51) Int. Cl. C07K 16/28; A61K 39/00; A61L 27/54; C07K 16/22

(52) U.S. Cl. CPC C07K 16/2863; A61L 27/54; C07K 16/22; A61K 2039/505; C07K 2317/31; C07K 2317/569; C07K 2317/76

(57) ABSTRACT

A BMP2 surrogate protein is provided comprising two different antigen binding regions (ABR), which can be configured as immunoglobulin “single variable domains” (ISV). A first ISV specifically binds to a first BMP2 receptor, e.g. BMPR1a; and a second ISV specifically binds to a second BMP2 receptor, e.g. BMPR2. In some embodiments the first and second ISV are joined by a cleavable linker that is susceptible to proteases selectively expressed in monocytic cells or macrophages, relative to skeletal stem cells.

### CROSS REFERENCE TO RELATED APPLICATION

The present application claims the benefit of and priority to U.S. Provisional Patent Application No. 63/196,540, filed Jun. 3, 2021, the entire disclosure of which is hereby incorporated by reference in its entirety.

### FEDERALLY SPONSORED RESEARCH AND DEVELOPMENT

This invention was made with Government support under contract AG049958 awarded by the National Institutes of Health. The Government has certain rights in the invention.

### INCORPORATION BY REFERENCE OF SEQUENCE LISTING PROVIDED AS A TEXT FILE

A Sequence Listing is provided here within in a text file, (S21-068_STAN-1844WO_SeqListing_ST25.txt), created on May 24, 2022, and having a size of 41,000 bytes.

### BACKGROUND

The most common type of musculoskeletal disorder is osteoarthritis (OA), which afflicts 15% of the adult population with a lifetime risk of 40%. Damaged articular cartilage has no ability to regenerate and extensive cartilage damage often necessitates invasive joint replacements. Stem cell therapy to regenerate damaged articular cartilage would be a highly attractive alternative to invasive joint replacement surgery. Recently, a study found an effective combination of BMP2 and VEGF antagonism using soluble VEGF receptor facilitated efficient regeneration of articular cartilage at OA joints after microfracture. These proteins target endogenous skeletal stem cells (SSCs) at the injury site and directed them towards cartilage differentiation. This BMP2-VEGFR protein therapy is very effective, however efficiency of the recombinant proteins is low, requiring high dosages, and BMP2 will activate monocyte and macrophages, causing inflammation and additional release of pro-inflammatory cytokines, resulting in pain and additional bone/cartilage resorption at the OA site.

The present disclosure addresses this problem.

### SUMMARY

Compositions and methods are provided for the activation of BMP2 signaling by a surrogate protein. A BMP2 surrogate protein of the present disclosure comprises two different antigen binding regions (ABR), which can be configured as immunoglobulin “single variable domains” (ISV). A first ISV specifically binds to a first BMP2 receptor, e.g. BMPR1a; and a second ISV specifically binds to a second BMP2 receptor, e.g. BMPR2. The surrogate protein can provide for enhanced activity relative to a wild-type BMP2 protein. In some embodiments the first and second ISV are joined by a cleavable linker that is susceptible to proteases selectively expressed in monocytic cells or macrophages, relative to skeletal stem cells. In some embodiments the linker is a polypeptide of from about 4 to about 40 amino acids in length, comprising a valine-citrulline (val-cit) moiety, which moiety is cleaved by elastase. The surrogate provides for high activity, targeted BMP2 delivery to tissues containing skeletal stem cells (SSC), with decreased activity on elastase expressing monocytes and macrophages.

In an embodiment, a first ISV of the BMP2 surrogate specifically binds to a specific region of BMPR1a exemplified by SEQ ID NO:1. In an embodiment, a second ISV of the BMP2 surrogate binds to human BMPR2 protein, for example binding to a polypeptide sequence exemplified by SEQ ID NO:2. As used herein, ISV is used as a general term to include but not be limited to antigen-binding domains or fragments such as V_{HH} domains or V_{H} or V_{L} domains, respectively.

In some embodiments, a first ISV of the BMP2 surrogate specifically binds to BMPR1a protein. In some such embodiments, the first ISV specifically binds to the polypeptide of SEQ ID NO:1. Exemplary ISV sequences for this purpose include, for example, those having the amino acid sequence of any of SEQ ID NO:4, also referred to as NB1-1; SEQ ID NO:8; SEQ ID NO:12, also referred to as NB1-4; SEQ ID NO:16, also referred to NB1-35; SEQ ID NO:20; SEQ ID NO:24; SEQ ID NO:28; or an ISV having the CDR1, CDR2 and CDR3 sequences thereof.

In some embodiments, a second ISV specifically binds to a BMPR2 protein. In some such embodiments, the second ISV specifically binds to the polypeptide of SEQ ID NO:2. Exemplary ISV sequences for this purpose include, for example, those binding to a human BMPR2 protein; and having the amino acid sequence of any of SEQ ID NO:32; SEQ ID NO:36; SEQ ID NO:40; SEQ ID NO:44; SEQ ID NO:48; SEQ ID NO:52, also referred to as NB2-43; SEQ ID NO:56; SEQ ID NO:60; SEQ ID NO:64; SEQ ID NO:64, also referred to as NB2-9; SEQ ID NO:68 or an ISV having the CDR1, CDR2 and CDR3 sequences thereof.

In some embodiments the BMP2 surrogate is administered at an effective dose for activation of SSC. The effective dose can be locally administered. In some embodiments, SSC are present at a site of injury, including without limitation an acute local injury, a site of osteoarthritis, a site of a bone or dental implant, a site of microfracture, etc. Exogenous SSC are optionally co-administered. In some embodiments the SSC are induced to regenerate bone. In some embodiments the SSC are induced to regenerate cartilage. The BMP2 surrogate may be co-administered with additional agents effective to guide the SSC to an osteogenic or chondrogenic fate. Appropriate activation of signaling pathways enhances activation and proliferation of SSC; and selectively guides cell differentiation.

In some embodiments, where regeneration of cartilage, particularly articular cartilage, is desired, an effective dose of the BMP2 surrogate is co-administered with an effective dose of a vascular endothelial growth factor (VEGF) antagonist as a second active agent. Localized delivery of a bone morphogenetic protein 2 (BMP2) surrogate and a VEGF antagonist collectively promotes a chondrogenic fate and leads to regeneration of cartilage.

In some embodiments a BMP2 surrogate is joined to a third ISV that antagonizes VEGF. In such an embodiment, the third ISV may bind to human VEGFA protein, for example binding to a polypeptide sequence represented by SEQ ID NO:3. Such a protein may be referred to as a “trispecific protein”.

In other embodiments, the BMP2 surrogate is co-administered or co-formulated with an effective dose of a VEGF antagonist as a second active agent. The dose of VEGF antagonist may vary with the specific agent, e.g. small molecule, antibody, soluble VEGFR1, etc. Examples of useful VEGF antagonists include, without limitation, ziv-aflibercept; bevacizumab; pazopanib; cabozantinib; sunitinib; sorafenib; axitinib; lenvatinib; regorafenib; ponatinib; cabozantinib; vandetanib; ramucirumab; bevacizumab; bevacizumab; and the like. In some embodiments the effective dose of VEGF antagonist in a single drug delivery device, e.g. an implant, is from about 0.1 μg to about 50 mg. For delivery to a human, the dose may be from about 0.1 μg, from about 0.5 μg, from about 1 μg, from about 25 μg, up to about 10 mg, up to about 5 mg, up to about 2.5 mg, up to about 1 mg.

The ISV domains of a BMP2 surrogate or trispecific protein may be joined through one or more polypeptide linkers. In an embodiment, the polypeptide linker is a protease susceptible, flexible linker of from about 4 to 30 amino acids in length. In some embodiments a polypeptide linker susceptible to proteases found in specifically in monocytes and macrophages is used, e.g. susceptible to serine proteases such as chymases, elastases, tryptases, aspases, and metases. In some embodiments, the protease sensitive linker comprises a Val-Cit moiety, which provides a site for cleavage by certain proteases. In some embodiments, the Val-Cit residues are positioned on a linker between the first and second ISV but not the third. In some embodiments the polypeptide linker is from about 8 to about 30 amino acids in length, e.g. from about 10 to about 30, from about 12 to about 30, from about 15 to 30; from about 15-25, from about 15-20 amino acids and is comprised of a poly-(gly-ser) sequence. A linker joining the two ISV domains of the BMP2 surrogate moiety may be the same or different as a peptide linker joining the BMP2 surrogate to the VEGF antagonist.

In some embodiments, the BMP2 surrogate or trispecific protein further comprises a TrxA sequence to facilitate folding.

In an embodiments, when the regeneration of bone is desired, a second active agent is co-administered with a BMP2 surrogate to enhance bone regeneration. In some embodiments, the second active agent is covalently linked to a BMP2 surrogate of the present disclosure through a cleavable or non-cleavable linker. Secondary active agents that find use in the present disclosure includes, without limitation, a WNT activator, a Hedgehog activator, a TGF-β pathway inhibitor, a SIRT1 activator, a ferroptosis inhibitor, a calcineurin inhibitor, a BMP4 agonist, a BMP pathway activator, and a VEGF agonist.

In some embodiments, the BMP2 surrogate, alone or in combination with a second active agent, is administered to a local site for regeneration of bone or cartilage in a drug delivery device, including without limitation a biodegradable implant. In some embodiments a drug delivery device is implanted at a site of local injury including without limitation an acute local injury, a site of osteoarthritis, a site of a bone or dental implant, a site of microfracture, etc. In some embodiments a BMP2 surrogate, alone or in combination with a second active agent, provides the sole active agents in a drug delivery device.

In some embodiments a drug delivery device for use in the methods described herein is provided. In some embodiments the drug delivery device comprises an effective dose of a BMP2 surrogate optionally in combination with an effective dose of second active agent, for example a VEGF inhibitor, e.g. in the form of a trispecific protein. In some embodiments the drug delivery device is a biocompatible hydrogel. In some embodiments the drug delivery device is a biodegradable hydrogel.

In some embodiments the effective dose of a BMP2 surrogate in a single drug delivery device, e.g. an implant, is from about 1 μg to about 50 mg. For delivery to a human, the dose may be from about 10 μg, from about 25 μg, from about 50 μg, from about 100 μg, up to about 50 mg, up to about 25 mg, up to about 10 mg, up to about 5 mg, up to about 1 mg.

In some embodiments the drug delivery device is implanted at the site of local injury in the absence of exogenous cells. In other embodiments, particularly in the treatment of aged individuals, for example humans over the age of 50, over the age of 60, over the age of 70, etc., an effective dose of skeletal or non-skeletal stem cells are provided with the active agents, where the skeletal or non-skeletal stem cells are optionally allogeneic.

In embodiments, methods are provided for regeneration of articular cartilage, or bone, at a targeted site in a subject in need thereof, where a drug delivery device is implanted at the targeted site, the device configured to release an effective dose of a BMP2 surrogate optionally in combination with an effective dose of a second active agent, including without limitation a VEGF inhibitor, e.g. in the form of a trispecific protein. The device may be implanted immediately following an injury, within 1 hour, within 2 hours, within 4 hours, within 6 hours, within 12 hours, within 1 day, within 2 days, within 3 days. The drug delivery device is optionally a biodegradable matrix, e.g. a hydrogel, that does not need to be removed following cartilage regeneration. The surgical site is closed, and function may be assessed after about 1 week, after about 2 weeks, after about 3 weeks, after about 4 week, after about 6 weeks, after about 8 weeks, or more, as needed, and may be assessed at multiple time points.

In some embodiments a local acute injury that generates mechanical activation of endogenous SSC is provided by a microfracture/drilling procedure to bone tissue at a desired site for cartilage regeneration. Microfracture (MF) is a minimally invasive surgery that promotes bleeding from a bone to create a clot in a cartilage defect. Following MF, there is a therapeutic window of time to skew MF-activated SSC differentiation fate towards robust formation of cartilage, e.g. immediately following the injury, within 1 hour, within 2 hours, within 4 hours, within 6 hours, within 12 hours, within 1 day, within 2 days, within 3 days. Combining microfracture with the provision of suitable growth factors for SSC proliferation and differentiation to cartilage within the therapeutic window provides for regeneration of stable articular cartilage at the site. The cartilage thus formed has substantially reduced levels of fibrocartilage, relative to the levels seen in the absence of the growth factors.

These and other objects, advantages, and features of the invention will become apparent to those persons skilled in the art upon reading the details of the subject methods and compositions as more fully described below.

### BRIEF DESCRIPTION OF THE DRAWINGS

FIG. 1. OA/MF defects with transplants of hydrogels with the indicated factors. From left to right, gross images (scale bars, 1 mm); representative pentachrome stains of the regenerate (scale bars, 500 μm); and higher magnification (scale bars, 100 μm) of the pentachrome stains. n=8 mice per group.

FIG. 2. Experimental flow for fabricating BMP2 surrogate

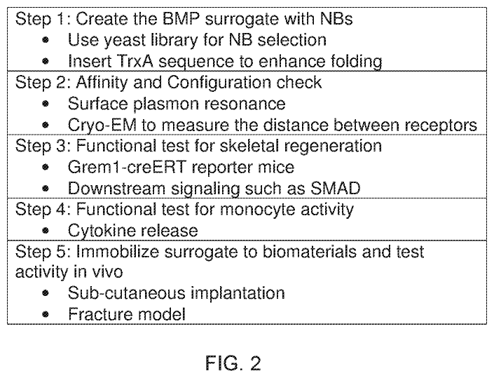

FIG. 3. Schematic illustration of our BMP2 surrogate targeting to skeletal stem cells. Upper: Controlling the distance between NB1 and NB2 allows us to optimize the BMP2 receptor signaling for better cartilage regeneration. Lower: If the surrogate binds to the receptor on monocyte, surrounding monocyte specific elastase will cleave the linker (Val-Cit) to disable the signaling.

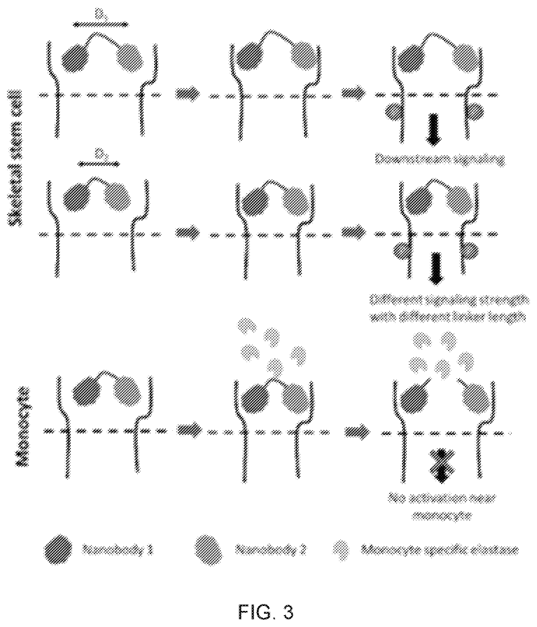

FIG. 4. Schematic of BMP2 signaling.

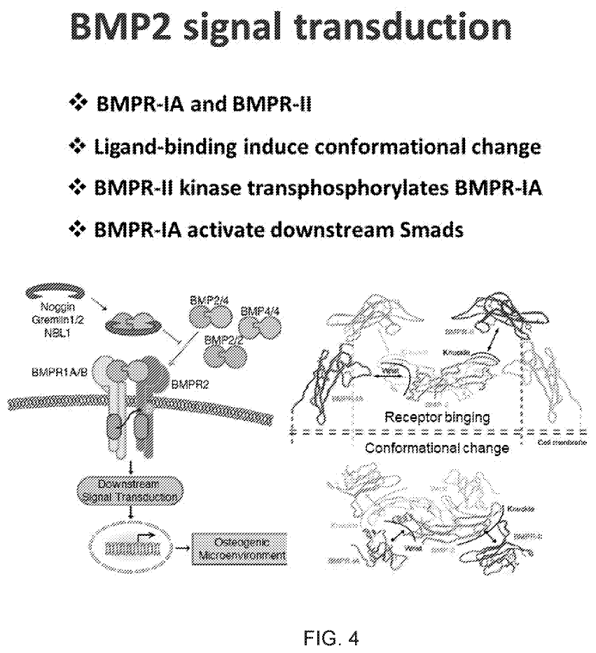

FIG. 5. Schematic of drug development process.

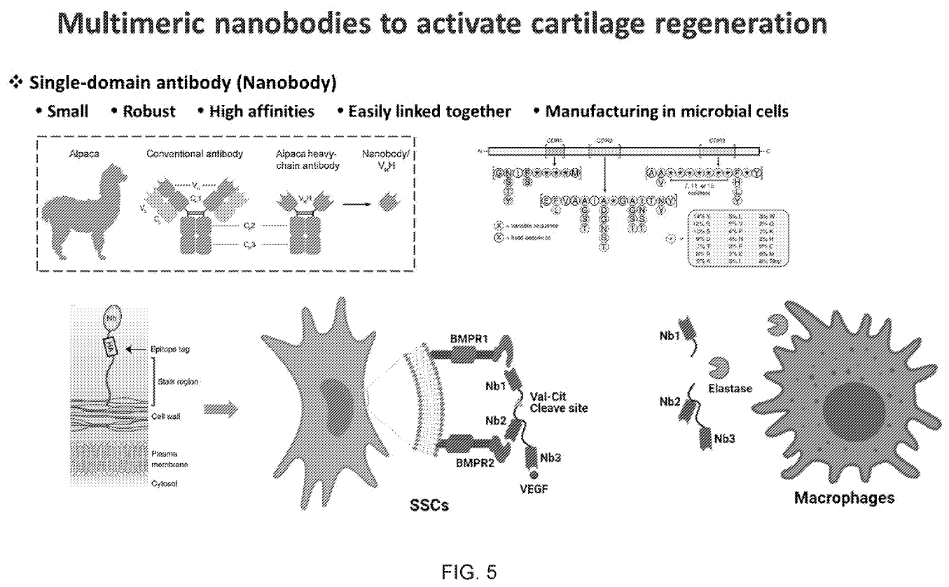

FIG. 6. (a) Western blot of fabricated BMPR1a and BMPR2. Both BMPR1a and BMPR2 have His and Flag tag. (b) After size chromatography, we separate monomer population from multimers. (c,d) Fabricated extracellular domain of BMPR1a and BMPR2 bind to BMP2 to block the downstream gene expression in skeletal stem cell. (e) p-smad activation in skeletal stem cell was examined after 30 minutes treatment of indicated amount of BMPR1a or BMPR2 together with BMP2.

FIG. 7. Skeletal stem cell was treated with 1) no treatment, 2) BMP2 (2.5 ng/ml), 3) BMP2 (2.5 ng/ml) and NB1 (25 ng/ml), 4) 5 minutes pre-treatment of NB1 (25 ng/ml) and BMP2 (2.5 ng/ml), 5) 5 minutes pre-treatment of NB1 (250 ng/ml) and BMP2 (2.5 ng/ml). P-smad 1/5 activation was examined 30 minutes after the BMP2 treatment.

FIG. 8. a) Skeletal stem cells were treated with BMP2 (2.5 ng/ml) or indicated combination of bispecific nanobody (250 ng/ml). P-smad activation was measured after 30 minutes treatment. b) Skeletal stem cells were treated with BMP2 (2.5 ng/ml) or indicated combination of unlinked nanobodies. P-smad activation was measured after 30 minutes treatment. c) P-smad band intensity of (a) was quantitatively characterized.

### DESCRIPTION OF THE SPECIFIC EMBODIMENTS

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

As used herein, the term “BMP2” refers to the family of bone morphogenetic proteins of the type 2, derived from any species, and may include mimetics and variants thereof. Reference to BMP2 herein is understood to be a reference to any one of the currently identified forms, including BMP2A and BMP2B, as well as to BMP2 species identified in the future. The term “BMP2” also includes polypeptides derived from the sequence of any known BMP2 whose mature sequence is at least about 75% homologous with the sequence of a mature human BMP2, which reference sequence may be found in Genbank, accession number NP_001191.

BMP2 signals via two types of receptors (BRI and BRII) that are expressed at the cell surface as homomeric as well as heteromeric complexes. Prior to ligand binding, a low but measurable level of BMP-receptors is found in preformed hetero-oligomeric complexes. The major fraction of the receptors is recruited into hetero-oligomeric complexes only after ligand addition. For this, BMP2 binds first to the high affinity receptor BRI and then recruits BRII into the signaling complex. However, ligand binding to the preformed complex composed of BRII and BRI is still required for signaling, suggesting that it may mediate activating conformational changes. Signals induced by binding of BMP2 to preformed receptor complexes activate the Smad pathway, whereas BMP2-induced recruitment of receptors activates a different, Smad-independent pathway resulting in the induction of alkaline phosphatase activity via p38 MAPK.

In some embodiments, a dose of a BMP2 surrogate is provided in an implant, e.g. a matrix or scaffold for localized delivery of the factor. The effective dose may be determined based on the specific tissue, rate of release from the implant, size of the implant, and the like. and may be empirically determined by one of skill in the art. The dose may provide for biological activity equivalent to 1 μg recombinant BMP2 protein, 10 μg, 100 jg, 1 mg, 5 mg, 10 mg, 25 mg, 50 mg, 75 mg, 100 mg, 250 mg, 500 mg, 750 mg, 1 g of BMP2 protein. The dose may be administered at a single time point, e.g. as a single implant; or may be fractionated, e.g. delivered in a microneedle configuration. The dose may be administered, once, two, three time, 4 times, 5 times, 10 times, or mare as required to achieve the desired effect, and administration may be daily, every 2 days, every 3 days, every 4 days, weekly, bi-weekly, monthly, or more.

VEGF is a dimeric, disulfide-linked 46-kDa glycoprotein related to Platelet-Derived Growth Factor (“PDGF”). It is produced by normal cell lines and tumor cell lines; is an endothelial cell-selective mitogen; shows angiogenic activity in in vivo test systems (e.g., rabbit cornea); is chemotactic for endothelial cells and monocytes; and induces plasminogen activators in endothelial cells, which are involved in the proteolytic degradation of the extracellular matrix during the formation of capillaries. A number of isoforms of VEGF are known, which while they show comparable biological activity, differ in the type of cells that secrete them and in their heparin-binding capacity. In addition, there are other members of the VEGF family, such as Placenta Growth Factor (“PGF”) and VEGF-C.

The cellular receptors of VEGFs (VEGFRs) are transmembranous receptor tyrosine kinases. They are characterized by an extracellular domain with seven immunoglobulin-like domains and an intracellular tyrosine kinase domain. Various types of VEGF receptor have been characterized, including VEGFR-1 (also known as flt-1), VEGFR-2 (also known as KDR), and VEGFR-3.

“VEGF inhibitor” or “VEGF antagonist” as used herein is any substance that decreases signaling by the VEGF-VEGFR pathway. VEGF inhibitors can be, to name just a few examples, small molecules, peptides, polypeptides, proteins, including more specifically antibodies, including anti-VEGF antibodies, anti-VEGFR antibodies, intrabodies, maxibodies, minibodies, diabodies, Fc fusion proteins such as peptibodies, receptibodies, soluble VEGF receptor proteins and fragments, and a variety of others. Many VEGF inhibitors work by binding to VEGF or to a VEGF receptor. Others work more indirectly by binding to factors that bind to VEGF or to a VEGF receptor or to other components of the VEGF signaling pathway. Still other VEGF inhibitors act by altering regulatory posttranslational modifications that modulate VEGF pathway signaling. VEGF inhibitors in accordance with the invention also may act through more indirect mechanisms. Whatever the mechanism involved, as used herein, a VEGF inhibitor decreases the effective activity of the VEGF signaling pathway in a given circumstance over what it would be in the same circumstance in the absence of the inhibitor.

In some embodiments, a dose of VEGF inhibitor is provided in an implant, e.g. a matrix or scaffold for localized delivery of the facto. The effective dose may be determined based on the specific tissue, rate of release from the implant, size of the implant, and the like. and may be empirically determined by one of skill in the art. The dose may provide for biological activity equivalent to 1 μg soluble VEGF receptor, 10 μg, 100 μg, 1 mg, 5 mg, 10 mg, 25 mg, 50 mg, 75 mg, 100 mg, 250 mg, 500 mg, 750 mg, 1 g of soluble VEGF receptor. The dose may be administered at a single time point, e.g. as a single implant; or may be fractionated, e.g. delivered in a microneedle configuration. The dose may be administered, once, two, three time, 4 times, 5 times, 10 times, or mare as required to achieve the desired effect, and administration may be daily, every 2 days, every 3 days, every 4 days, weekly, bi-weekly, monthly, or more.

A great many VEGF inhibitors have been described in the literature. In addition to those described in further detail below, VEGF inhibitors are described in the following patent documents: US 2003/0105091, US2006/0241115, U.S. Pat. Nos. 5,521,184, 5,770,599, 5,990,141 U.S. Pat. Nos. 6,235,764, 6,258,812, 6,515,004, 6,630,500, 6,713,485, WO2005/070891 WO 01/32651, WO 02/68406, WO 02/66470, WO 02/55501, WO 04/05279, WO 04/07481, WO 04/07458, WO 04/09784, WO 02/59110, WO 99/450029, WO 00/59509, WO 99/61422, WO 00/12089, WO 00/02871, and WO 01/37820, particularly in parts pertinent to VEGF inhibitors.

The following are among specific VEGF inhibitors: ABT-869 (Abbott) including formulations for oral administration and closely related VEGF inhibitors; AEE-788 (Novartis) (also called AE-788 and NVP-AEE-788, among others) including formulations for oral administration and closely related VEGF inhibitors; AG-13736 (Pfizer) (also called AG-013736) including formulations for oral administration and closely related VEGF inhibitors; AG-028262 (Pfizer) and closely related VEGF inhibitors; Angiostatin (EntreMed) (also called CAS Registry Number 86090-08-6, K1-4, and rhuAngiostatin, among others) and closely related inhibitors as described in, among others, U.S. Pat. Nos. 5,792,825 and 6,025,688, particularly in parts pertaining to Angiostatin and closely related VEGF inhibitors, their structures and properties, and methods for making and using them; Avastin™ (Genentech) (also called bevacizumab, R-435, rhuMAB-VEGF, and CAS Registry Number 216974-75-3, among others) and closely related VEGF inhibitors; AVE-8062 (Ajinomoto Co. and Sanofi-aventis) (also called AC-7700 and combretastatin A4 analog, among others), and closely related VEGF inhibitors; AZD-2171 (AstraZeneca) and closely related VEGF inhibitors; Nexavar® (Bayer AG and Onyx) (also called CAS Registry Number 284461-73-0, BAY-43-9006, raf kinase inhibitor, sorafenib, sorafenib analogs, and IDDBCP150446, among others) and closely related VEGF inhibitors; BMS-387032 (Sunesis and Bristol-Myers Squibb) (also called SNS-032 and CAS Registry Number 345627-80-7, among others) and closely related VEGF inhibitors; CEP-7055 (Cephalon and Sanofi-aventis) (also called CEP-11981 and SSR-106462, among others) and closely related VEGF inhibitors; CHIR-258 (Chiron) (also called CAS Registry Number 405169-16-6, GFKI, and GFKI-258, among others) and closely related VEGF inhibitors; CP-547632 (OSI Pharmaceuticals and Pfizer) (also called CAS Registry Number 252003-65-9, among others) and closely related VEGF inhibitors such as, for instance, CP-564959; E-7080 (Eisai Co.) (also called CAS Registry Number 417716-92-8 and ER-203492-00, among others) and closely related VEGF inhibitors; 786034 (GlaxoSmithKline) and closely related VEGF inhibitors; GW-654652 (GlaxoSmithKline) and closely related indazolylpyrimidine Kdr inhibitors; IMC-1C11 (ImClone) (also called DC-101 and c-piC11, among others) and closely related VEGF inhibitors; KRN-951 (Kirin Brewery Co.) and other closely related quinoline-urea VEGF inhibitors; PKC-412 (Novartis) (also called CAS Registry Number 120685-11-2, benzoylstaurosporine, CGP-41251, midostaurin, and STI-412, among others) and closely related VEGF inhibitors; PTK-787 (Novartis and Schering) (also called CAS Registry Numbers 212141-54-3 and 212142-18-2, PTK/ZK, PTK-787/ZK-222584, ZK-22584, VEGF-TKI, VEGF-RKI, PTK-787A, DE-00268, CGP-79787, CGP-79787D, vatalanib, ZK-222584, among others) and closely related anilinophthalazine derivative VEGF inhibitors; SU11248 (Sugen and Pfizer) (also called SU-11248, SU-011248, SU-11248J, Sutent®, and sunitinib malate, among others) and closely related VEGF inhibitors; SU-5416 (Sugen and Pfizer/Pharmacia) (also called CAS Registry Number 194413-58-6, semaxanib, 204005-46-9, among others) and closely related VEGF inhibitors; SU-6668 (Sugen and Taiho) (also called CAS Registry Number 252916-29-3, SU-006668, and TSU-68, among others) and closely related VEGF inhibitors as described in, among others, WO-09948868, WO-09961422, and WO-00038519, particularly in parts pertaining to SU-6668 and closely related VEGF inhibitors, their structures and properties, and methods for making and using them; VEGF Trap (Regeneron and Sanofi-aventis) (also called AVE-0005 and Systemic VEGF Trap, among others) and closely related VEGF inhibitors as described in, among others, WO-2004110490, particularly in parts pertaining to VEGF Trap and closely related VEGF inhibitors, their structures and properties, and methods for making and using them; Thalidomide (Celgene) (also called CAS Registry Number 50-35-1, Synovir, Thalidomide Pharmion, and Thalomid, among others) and closely related VEGF inhibitors; XL-647 (Exelixis) (also called EXEL-7647, among others) and closely related VEGF inhibitors; XL-999 (Exelixis) (also called EXEL-0999, among others) and closely related VEGF inhibitors; XL-880 (Exelixis) (also called EXEL-2880, among others) and closely related VEGF inhibitors; ZD-6474 (AstraZeneca) (also called CAS Registry Number 443913-73-3, Zactima, and AZD-6474, among others) and closely related anilinoquinazoline VEGF inhibitors; and ZK-304709 (Schering) (also called CDK inhibitors (indirubin derivatives), ZK-CDK, MTGI, and multi-target tumor growth inhibitor, among others) and other closely related compounds including the indirubin derivative VEGF inhibitors described in WO-00234717, WO-02074742, WO-02100401, WO-00244148, WO-02096888, WO-03029223, WO-02092079, and WO-02094814, particularly in parts pertinent to these and closely related VEGF inhibitors, their structures and properties, and methods for making and using them.

VEGF inhibitors may be delivered in a manner appropriate to the nature of the inhibitor, e.g. as a protein, small molecule, nucleic acid, etc., including without limitation appropriate vehicles and vectors as required.

Immunoglobulin sequences, such as antibodies and antigen binding fragments derived there from (e.g., immunoglobulin single variable domains or ISVs) are used to specifically target the respective antigens disclosed herein. The generation of immunoglobulin single variable domains such as e.g., V_{HH}s or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

Alternatively, similar immunoglobulin single variable domains can be generated and selected by the immunization of an experimental animal such as a llama, construction of phage libraries from immune tissue, and

Unless indicated otherwise, the term “immunoglobulin single variable domain” or “ISV” is used as a general term to include but not limited to antigen-binding domains or fragments such as V_{HH} domains or V_{H} or V_{L} domains, respectively. V_{HH} domains are of interest for the present disclosure. The terms antigen-binding molecules or antigen-binding protein are used interchangeably and include also the term NANOBODIES®. The immunoglobulin single variable domains can be light chain variable domain sequences [e.g., a V_{L}-sequence), or heavy chain variable domain sequences (e.g., a V_{H}-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be single domain antibodies, or immunoglobulin sequences that are suitable for use as single domain antibodies, “dAbs”, or immunoglobulin sequences that are suitable for use as dAbs, or NANOBODIES™, including but not limited to V_{HH} sequences.

The invention includes immunoglobulin sequences of different origin, comprising mouse, rat, rabbit, donkey, human and camelid immunoglobulin sequences. The immunoglobulin single variable domain includes fully human, humanized, otherwise sequence optimized or chimeric immunoglobulin sequences. The immunoglobulin single variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. It is noted that the terms Nanobody or Nanobodies are registered trademarks of Ablynx N.V. and thus may also be referred to as NANOBODY® or NANOBODIES®, respectively.

An amino acid sequence such as e.g. an immunoglobulin single variable domain or polypeptide according to the invention is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, V_{HH} domains from Camelids in the article of Riechmann and Muyldermans, J. Immunol. Methods 2000 Jun. 23; 240 (1-2): 185-195. The CDR sequences of exemplary VHH domains are disclosed, along with the relevant CDR sequences in, for example, any of SEQ ID NO: 5-7,9-11, 13-15, 17-19, 21-23, 25-27, 29-31, 33-35, 37-39, 41-43, 45-47, 49-51, 53-55, 57-59, 61-63, or 65-67.

The present invention relates to particular polypeptides, also referred to as “polypeptides of the invention” that comprise or essentially consist of (i) a first building block consisting essentially of a first immunoglobulin single variable domain, (ii) a second building block consisting essentially of a second immunoglobulin single variable domain, and optionally (iii) a third building block consisting essentially of a third immunoglobulin single variable domain, linked via a linker.

Such immunoglobulin single variable domains may be derived in any suitable manner and from any suitable source, and may for example be naturally occurring V_{HH} sequences (i.e., from a suitable species of Camelid, e.g., llama) or synthetic or semi-synthetic VHs or VLs (e.g., from human). Such immunoglobulin single variable domains may include “humanized” or otherwise “sequence optimized” VHHs, “camelized” immunoglobulin sequences (and in particular camelized heavy chain variable domain sequences, i.e., camelized VHs), as well as human VHs, human VLs, camelid VH Hs that have been altered by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein.

Immunoglobulin single variable domains may comprise an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized immunoglobulin single variable domains of the invention can be obtained in any suitable manner known per se and thus are not strictly limited to polypeptides that have been obtained using a polypeptide that comprises a naturally occurring V_{HH} domain as a starting material.

Another class of immunoglobulin single variable domains of the invention comprises immunoglobulin single variable domains with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the description herein. Such “camelizing” substitutions may be inserted at amino acid positions that form and/or are present at the V_{H}-V_{L} interface, and/or at the so-called Camelidae hallmark residues (see for example WO 94/04678 and Davies and Riechmann (1994 and 1996)). The V_{H} sequence that is used as a starting material or starting point for generating or designing the camelized immunoglobulin single variable domains is preferably a V_{H} sequence from a mammal, more preferably the V_{H} sequence of a human being, such as a V_{H}3 sequence. However, it should be noted that such camelized immunoglobulin single variable domains of the invention can be obtained in any suitable manner known per se and thus are not strictly limited to polypeptides that have been obtained using a polypeptide that comprises a naturally occurring V_{H} domain as a starting material.

The term “antibody” herein is used in the broadest sense and specifically covers monoclonal antibodies, polyclonal antibodies, monomers, dimers, multimers, multispecific antibodies (e.g., trispecific antibodies), heavy chain only antibodies, three chain antibodies, single chain Fv, single domain antibodies, nanobodies, etc., and also include antibody fragments with or without pegylation, so long as they exhibit the desired biological activity (Miller et al (2003) Jour. of Immunology 170:4854-4861).

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or phagocytosis. A functional antibody may also block ligand activation of a receptor or act as an agonist or antagonist or as an allosteric modulator.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof.

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; nanobodies or domain antibodies comprising single Ig domains from human or non-human species or other specific single-domain binding modules including non-antibody binding proteins such as, but not limited to, adnectins and anticalins; and multispecific or multivalent structures formed from antibody fragments.

The term “NANOBODY®” as used herein refers to a single domain antibody consisting of a single monomeric variable domain (also referred to as a variable heavy homodimer [V_{HH}] domain). The single domain antibodies are naturally produced by animals belonging to the camelid family. Nanobodies are smaller than human antibodies, where ISV are generally 12-15 kDa, human antibodies are generally 150-160 kDa, Fab fragments are −50 kDa and single-chain variable fragments are −25 kDa. NANOBODIES® provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like.

Linker. The binding domains of a BMP2 surrogate, trispecific protein, or multispecific protein may be separated by a linker, e.g. a polypeptide linker, or a non-peptidic linker, etc. The amino acid linkers that join domains can play an important role in the structure and function of multi-domain proteins. The length of the linker and therefore the spacing between the binding domains, can be used to modulate the signal strength of the surrogate, and can be selected depending on the desired use of the surrogate. The enforced distance between binding domains of a surrogate can vary, but in certain embodiments may be less than about 100 angstroms, less than about 90 angstroms, less than about 80 angstroms, less than about 70 angstroms, less than about 60 angstroms, less than about 50 angstroms, less than about 40 angstroms, less than about 30 angstroms, less than about 20 angstroms.

In some embodiments, a polypeptide linker is used, which linker comprises at least one val-cit moiety. Certain proteases, including elastase, cleaves the peptide bond at the citrulline of valine (Val)-citrulline (Cit), thereby separating the two ISV of the surrogate, and reducing activity of the surrogate in cells expressing such proteases. The val-cit moiety has a structure:

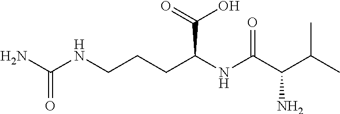 [SMILES: CC(C)[C@H](N)C(=O)N[C@@H](CCCNC(N)=O)C(=O)O]

In some embodiments the linker is a rigid linker, in other embodiments the linker is a flexible linker. In some embodiments, the linker moiety is a peptide linker. In some embodiments, the peptide linker comprises from about 4 to about 30 amino acids. In some embodiments, the peptide linker comprises 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids, but no greater than 100 amino acids. In some embodiments, the peptide linker is between 4 to 30, 5 to 25, 5 to 20, 5 to 15, 5 to 10 or 5 to 9 amino acids in length. Exemplary linkers include linear peptides having at least two amino acid residues such as Gly-Gly, Gly-Ala-Gly, Gly-Gly-Gly-Gly-Ser. Suitable linear peptides include poly glycine, polyserine, polyproline, polyalanine and oligopeptides consisting of alanyl and/or serinyl and/or prolinyl and/or glycyl amino acid residues. In one embodiment a linker comprises the amino acid sequence GSTSGSGKSSEGKG, or (GGGGS)n, where n is 1, 2, 3, 4, 5, etc.; however many such linkers are known and used in the art and may serve this purpose.

Chemical groups that find use in linking binding domains include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

The linkage between binding domains may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O)n where n is from 1 to about 12; (CH₂CH₂NH)n, where n is from 1 to about 12; [(CH₂)n(C═O)NH(CH₂)ₘ]_{z}, where n and m are from 1 to about 6, and z is from 1 to about 10; [(CH₂)nOPO₃(CH₂)ₘ]_{z} where n and m are from 1 to about 6, and z is from 1 to about 10. Such linkers may include polyethylene glycol, which may be linear or branched.

The active agents, e.g. the BMP2 surrogate and a second active agent that enhances bone or cartilage regeneration, may be joined through a homo- or heterobifunctional linker having a group at one end capable of forming a stable linkage to the hydrophilic head group, and a group at the opposite end capable of forming a stable linkage to the targeting moiety. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N, N′-(1,3-phenylene) bismaleimide; N, N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

Drug delivery devices include structures that can be implanted and that release the active agents, e.g. a BMP2 surrogate alone or in combination with a VEGF antagonist, e.g. in the form of a trispecific protein, at the targeted site. Implantable drug delivery devices can be broadly classified in two main groups: passive implants and active implants. The first group includes two main types of implants: biodegradable and non-biodegradable implants. Active systems rely on energy dependent methods that provide the driving force to control drug release. The second group includes devices such as osmotic pressure gradients and electromechanical drives.

Passive Polymeric Implants are normally relatively simple devices with no moving parts, they rely on passive diffusion for drug release. They are generally made of drugs packed within a biocompatible polymer molecule. Several parameters such as: drug type/concentration, polymer type, implant design and surface properties can be modified to control the release profile. Passive implants can be classified in two main categories: non-biodegradable and biodegradable systems.

Non-biodegradable implants are commonly prepared using polymers such as silicones, poly(urethanes), poly(acrylates) or copolymers such as poly(ethyelene vinyl acetate). Poly(ethylene-vinyl acetate) (PEVA) is a thermoplastic copolymer of ethylene and vinyl acetate. Poly(siloxanes) or silicones are organosilicon polymeric materials composed of silicon and oxygen atoms. Lateral groups can be methyl, vinyl or phenyl groups. These groups will influence the properties of the polymer. Poly(siloxanes) have been extensively used in medicine due to the unique combination of thermal stability, biocompatibility, chemical inertness and elastomeric properties. The silicones commonly used for medical devices are vulcanised at room temperature. They are prepared using a two-component poly(dimethylsiloxanes) (PDMS) in the presence of a catalyst (platinum based compound). The final material is formed via an addition hydrosilation reaction. An alternative method to obtain silicones for medical applications is the using linear PDMS with hydroxyl terminal groups. This linear polymer is cross-linked with low molecular weight tetra(alkyloxysilane) using stannous octoate catalyst.

This type of device can be monolithic or reservoir type implant. Monolithic type implants are made from a polymer matrix in which the drug is homogeneously dispersed. On the other hand, reservoir-type implants contain a compact drug core covered by a permeable non-biodegradable membrane. The membrane thickness and the permeability of the drug through the membrane will govern the release kinetics.

Biodegradable implants are made using polymers or block copolymers that can be broken down into smaller fragments that will be subsequently excreted or absorbed by the body. Normally they are made using polymers such as collagen, PEG, chitin, poly(caprolactone) (PCL), poly(lactic acid) (PLA) or poly(lactic-co-glycolic acid) (PLGA). Numerous other biodegradable polymers for drug delivery exist including: poly(amides), poly(anhydrides), poly(phosphazenes) and poly(dioxanone). Poly(anhydrides) have a low hydrolytic stability resulting in rapid degradation rates, making them suitable for use in short-term controlled delivery systems. Poly(phosphazenes) have a degradation rate that can be finely tuned by appropriate substitution with specific chemical groups and use of these polymers has been investigated for skeletal tissue regeneration and drug delivery. Poly(dioxanone), like PCL, is a polylactone that has been used for purposes such as drug delivery, and tissue engineering They do not need to be extracted after implantation, as they will be degraded by the body of the patient. They can be manufactured as monolithic implants and reservoir-type implants. In addition to the biopolymers, such as the abovementioned PLA, there a few natural polymers which also represent a promising class of materials with a wide range of applications, including use in implantable devices. These natural polymers include, collagen, hyaluronic acid, cellulose, chitosan, silk and others naturally derived proteins, as well as collagen, gelatin, albumin, elastin and milk proteins. These materials present certain advantages compared to the traditional materials (metals and ceramics) or synthetic polymers, such as biocompatibility, biodegradation and non-cytotoxicity, which make them ideal to be used in implantable drug delivery devices.

Dynamic or Active Polymeric Implants have a positive driving force to control the release of drugs from the device. The majority of the implants in this category are electronic systems made of metallic materials. Dynamic drug delivery implants are mainly pump type implants. The main type of polymeric active implants are osmotic pumps. This type of device is formed mainly by a semipermeable membrane that surrounds a drug reservoir. The membrane should have an orifice that will allow drug release. Osmotic gradients will allow a steady inflow of fluid within the implant. This process will lead to an increase in the pressure within the implant that will force drug release trough the orifice. This design allows constant drug release (zero order kinetics). This type of device allows a favorable release rate but the drug loading is limited.

In some embodiments, the factors are prepared as an injectable paste. The paste can be injected into the implant site. In some embodiments, the paste can be prepared prior to implantation and/or store the paste in the syringe at sub-ambient temperatures until needed. In some embodiments, application of the composite by injection can resemble a bone cement that can be used to join and hold bone fragments in place or to improve adhesion of, for example, a hip prosthesis, for replacement of damaged cartilage in joints, and the like. Implantation in a non-open surgical setting can also be performed.

In other embodiments the factors are prepared as formable putty. The hydrated graft putty can be prepared and molded to approximate any implant shape. The putty can then be pressed into place to fill a void in the cartilage, bone, tooth socket or other site. In some embodiments, graft putty can be used to repair defects in non-union bone or in other situations where the fracture, hole or void to be filled is large and requires a degree of mechanical integrity in the implant material to both fill the gap and retain its shape.

A system for pharmaceutical use, i.e. a drug delivery device with factors, can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the NR pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical composition, i.e. combinations of factors and/or cells, can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

The term “skeletal stem cell” refers to a multipotent and self-renewing cell capable of generating bone marrow stromal cells, skeletal cells, and chondrogenic cells. By self-renewing, it is meant that when they undergo mitosis, they produce at least one daughter cell that is a skeletal stem cell. By multipotent it is meant that it is capable of giving rise to progenitor cell (skeletal progenitors) that give rise to all cell types of the skeletal system. They are not pluripotent, that is, they are not capable of giving rise to cells of other organs in vivo.

Skeletal stem cells can be reprogrammed from non-skeletal cells, including without limitation mesenchymal stem cells, and adipose tissue containing such cells, such as human adipose stem cells (hAASC). Induced skeletal cells have characteristics of functional SSCs derived from nature, that is, they can give rise to the same lineages.

Human SSC cell populations may be characterized by their cell surface markers, although it will be understood by one of skill in the art that endogenous populations of SSC need not be characterized for effective stimulation. Human SSC are negative for expression of CD45, CD235, Tie2, and CD31; and positively express podoplanin (PDPN). A population of cells, e.g. cells isolated from bone tissue, having this combination of markers may be referred to as [PDPN⁺/146⁻] cells. The [PDPN⁺/146⁻] population can be further subdivided into three populations: a unipotent subset capable of chondrogenesis [PDPN⁺CD146⁻CD73⁻CD164⁻], a unipotent cellular subpopulation capable of osteogenesis [PDPN⁺CD146⁻CD73⁻CD164⁺] and a multipotent [PDPN⁺CD146⁻CD73+CD164⁺] cell capable of endochondral (bone and cartilage) ossification. A population of cells of interest for use in the methods of the invention may be isolated from bone with respect to CD45, CD235, Tie2, and CD31 and PDPN. Other cell populations of interest are [PDPN⁺CD146⁻CD73⁻CD164⁻] cells; [PDPN⁺CD146⁻CD73⁻CD164⁺] cells; and [PDPN⁺CD146⁻CD73+CD164⁺] cells.

The mouse skeletal lineage is characterized as CD45⁻, Ter119⁻, Tie2⁻, αv integrin+. The SSC is further characterized as Thy1− 6C3− CD105− CD200+.

By “proliferate” it is meant to divide by mitosis, i.e. undergo mitosis. An “expanded population” is a population of cells that has proliferated, i.e. undergone mitosis, such that the expanded population has an increase in cell number, that is, a greater number of cells, than the population at the outset.

The term “tissue” refers to a group or layer of similarly specialized cells which together perform certain special functions.

The term “organ” refers to two or more adjacent layers of tissue, which layers of tissue maintain some form of cell-cell and/or cell-matrix interaction to form a microarchitecture.

The terms “individual,” “subject,” “host,” and “patient,” are used interchangeably herein and refer to any mammalian subject for whom diagnosis, treatment, or therapy is desired, particularly humans.

Mammalian species that may be treated with the present methods include canines and felines; equines; bovines; ovines; etc. and primates, particularly humans. Animal models, particularly small mammals, e.g. murine, lagomorpha, etc. may be used for experimental investigations.

More particularly, the present invention finds use in the treatment of subjects, such as human patients, in need of bone or cartilage replacement therapy. Examples of such subjects are subjects suffering from conditions associated with the loss of cartilage from osteoarthritis, genetic defects, disease, etc. Patients having diseases and disorders characterized by such conditions can benefit greatly by a treatment protocol of the pending claimed invention.

An effective amount of a pharmaceutical composition of the invention is the amount that will result in an increase the cartilage regeneration or bone regeneration at the site of implant. For example, an effective amount of a pharmaceutical composition will increase cartilage, or bone, mass by at least about 5%, at least about 10%, at least about 20%, preferably from about 20% to about 50%, and even more preferably, by greater than 50% (e.g., from about 50% to about 100%) as compared to the appropriate control, the control typically being a subject not treated with the composition.

The methods of the present invention also find use in combined therapies, e.g. in with therapies that are already known in the art to provide relief from symptoms associated with the aforementioned diseases, disorders and conditions. The combined use of a pharmaceutical composition of the present invention and these other agents may have the advantages that the required dosages for the individual drugs is lower, and the effect of the different drugs complementary.

Articular cartilage. Articular cartilage is the highly specialized connective tissue of diarthrodial joints. Its principal function is to provide a smooth, lubricated surface for articulation and to facilitate the transmission of loads with a low frictional coefficient. Articular cartilage is devoid of blood vessels, lymphatics, and nerves and is subject to a harsh biomechanical environment. Most important, articular cartilage has a limited capacity for intrinsic healing and repair. In this regard, the preservation and health of articular cartilage are paramount to joint health.

The surfaces of articulating bones in mammalian joints are covered with articular cartilage. The articular cartilage prevents direct contact of the opposing bone surfaces and permits the near frictionless movement of the articulating bones relative to one another. Two types of articular cartilage defects are commonly observed in mammals and include full-thickness and partial-thickness defects. The two-types of defects differ not only in the extent of physical damage but also in the nature of repair response each type of lesion elicits.

Full-thickness articular cartilage defects include damage to the articular cartilage, the underlying subchondral bone tissue, and the calcified layer of cartilage located between the articular cartilage and the subchondral bone. Full-thickness defects typically arise during severe trauma of the joint or during the late stages of degenerative joint diseases, for example, during osteoarthritis. Since the subchondral bone tissue is both innervated and vascularized, damage to this tissue is often painful. The repair reaction induced by damage to the subchondral bone usually results in the formation of fibrocartilage at the site of the full-thickness defect. Fibrocartilage, however, lacks the biomechanical properties of articular cartilage and fails to persist in the joint on a long term basis.

Partial-thickness articular cartilage defects are restricted to the cartilage tissue itself. These defects usually include fissures or clefts in the articulating surface of the cartilage. Partial-thickness defects are caused by mechanical arrangements of the joint which in turn induce wearing of the cartilage tissue within the joint. In the absence of innervation and vasculature, partial-thickness defects do not elicit repair responses and therefore tend not to heal. Although painless, partial-thickness defects often degenerate into full-thickness defects.

Articular cartilage is hyaline cartilage and is 2 to 4 mm thick. It is composed of a dense extracellular matrix (ECM) with a sparse distribution of chondrocytes The ECM is principally composed of water, collagen, and proteoglycans, with other noncollagenous proteins and glycoproteins present in lesser amounts. Along with collagen fiber ultrastructure and ECM, chondrocytes contribute to the various zones of articular cartilage—the superficial zone, the middle zone, the deep zone, and the calcified zone. Within each zone, 3 regions can be identified—the pericellular region, the territorial region, and the interterritorial region.

The thin superficial (tangential) zone protects deeper layers from shear stresses and makes up approximately 10% to 20% of articular cartilage thickness. The collagen fibers of this zone (primarily, type II and IX collagen) are packed tightly and aligned parallel to the articular surface. The superficial layer contains a relatively high number of flattened chondrocytes, and the integrity of this layer is imperative in the protection and maintenance of deeper layers. This zone is in contact with synovial fluid and is responsible for most of the tensile properties of cartilage, which enable it to resist the sheer, tensile, and compressive forces imposed by articulation.

Immediately deep to the superficial zone is the middle (transitional) zone, which provides an anatomic and functional bridge between the superficial and deep zones. The middle zone represents 40% to 60% of the total cartilage volume, and it contains proteoglycans and thicker collagen fibrils. In this layer, the collagen is organized obliquely, and the chondrocytes are spherical and at low density. Functionally, the middle zone is the first line of resistance to compressive forces.

The deep zone is responsible for providing the greatest resistance to compressive forces, given that collagen fibrils are arranged perpendicular to the articular surface. The deep zone contains the largest diameter collagen fibrils in a radial disposition, the highest proteoglycan content, and the lowest water concentration. The chondrocytes are typically arranged in columnar orientation, parallel to the collagen fibers and perpendicular to the joint line. The deep zone represents approximately 30% of articular cartilage volume.

The tide mark distinguishes the deep zone from the calcified cartilage. The deep zone is responsible for providing the greatest amount of resistance to compressive forces, given the high proteoglycan content. Of note, the collagen fibrils are arranged perpendicular to the articular cartilage. The calcified layer plays an integral role in securing the cartilage to bone, by anchoring the collagen fibrils of the deep zone to subchondral bone. In this zone, the cell population is scarce and chondrocytes are hypertrophic.

Collagen is the most abundant structural macromolecule in ECM, and it makes up about 60% of the dry weight of cartilage. Type II collagen represents 90% to 95% of the collagen in ECM and forms fibrils and fibers intertwined with proteoglycan aggregates. Collagen types I, IV, V, VI, IX, and XI are also present but contribute only a minor proportion. The minor collagens help to form and stabilize the type II collagen fibril network.

Proteoglycans account for 10% to 15% of the wet weight of cartilage. Articular cartilage contains a variety of proteoglycans that are essential for normal function, including aggrecan, decorin, biglycan, and fibromodulin. The largest in size and the most abundant by weight is aggrecan.

Chondrocytes are the resident cell type in articular cartilage. Chondrocytes are highly specialized, metabolically active cells that play a unique role in the development, maintenance, and repair of the ECM. Chondrocytes have limited potential for replication, a factor that contributes to the limited intrinsic healing capacity of cartilage in response to injury. Chondrocyte survival depends on an optimal chemical and mechanical environment. Biochemical markers of chondrocytes, include without limitation, collagen type II, chondroitin sulfate, keratin sulfate and characteristic morphologic markers of smooth muscle, including but not limited to the rounded morphology observed in culture, and able to secrete collagen type II, including but not limited to the generation of tissue or matrices with hemodynamic properties of cartilage in vitro.

Fibrocartilage. Fibrocartilage is formed by acute local injury at a bone site in the absence of biochemical factors to direct cartilage formation. The mechanical properties are inferior to articular cartilage. For example, indicia of fibrocartilage include proteoglycan-producing chondrocytes and fibrotic cells, which stain positive for collagen (COL) 1 and matrix metalloproteinase (MMP) 13 and negative for COL 2.

Microfracture. Microfracture is a surgical technique that has been developed to treat chondral defects, which are damaged areas of articular cartilage of the knee. It is a common procedure used to treat patients with full thickness damage to the articular cartilage that goes all the way down to the bone. Microfracture has been used in joints including, without limitation, shoulder, hip, ankle and knee.

A microfracture is performed as part of arthroscopic surgery. The area undergoing microfracture is prepared by removing any loose or damaged cartilage. Ideally, the area undergoing microfracture will be less than about 2 centimeters in diameter and have good, healthy surrounding cartilage. Then, a small, sharp pick (awl) or drill is used to create the small microfracture holes in the bone. The number of microfractures created depends on the size of the joint are being treated. Most patients with a 1- to 2-centimeter area of damage require five to 15 small microfracture holes in the bone. The penetration of the outer layers of bone allows blood and stem cells to form a clot in the area of the cartilage defect.

Microfracture is a surgical technique that produces “microfractures” in subchondral bone perpendicular to the surface. This technique may use various angled awls or “pics.”, or small drills. A rough, raw surface that could hold the clot may also be formed. The pic was ideal for this, as it produced fracture fragments that attracted and held the clot. In order for tissue to regenerate, cells must be present. In this procedure, the controlled “microfractures” through the subchondral bone allow access to marrow-based progenitor cells and growth factors. A marrow clot is formed at the base of a prepared chondral lesion. The pluripotent cells proliferate and differentiate.

General indications for microfracture include full-thickness defects, unstable cartilage that overlies the subchondral bone, and a partial-thickness lesion that, when probed, the cartilage simply scrapes off down to bone. Patient age is not a specific contraindication. While patients under 35 years of age have greater improvement, older patients still show improvement. The size of the lesion is also not a contraindication for microfracture. Lesions may less than 400 mm² or more than 400 mm². The height of the cartilage rim surrounding the lesion may be adequate to hold the clot in place.

MRI can be used to assess the thickness of the cartilage and determine other associated injuries. The MRI enables imaging of morphological changes such as chondral fibrillation, fissuring, focal defects and corresponding fragments, and more diffuse thinning and wear, all manifesting as changes of the chondral thickness and surface at the cartilage interface to joint fluid and synovium. Earlier chondral degenerative changes, such as softening or blistering, to later fibrotic change can also be visible as intrasubstance areas of MRI signal change and heterogeneity, although such evaluation is still qualitative in standard clinical practice.

A thorough diagnostic arthroscopic examination of the joint can be performed through 3 portals (inflow cannula, arthroscope, and working instruments). Particular attention is paid to anterior interval scarring, plicae, and the lateral retinaculum, which have the potential to increase compression between cartilage surfaces. Microfracture is the final intra-articular procedure performed. This allows the initial clot in the microfracture site to be preserved. This can also prevent loss of visualization with blood and fat droplets entering the knee from the microfracture.

After identification of the full-thickness articular cartilage lesion, all remaining unstable cartilage is removed. A hand-held curved curette and a full radius resector can be used to remove the loose or marginally attached cartilage back to a stable rim of cartilage. The calcified cartilage layer that remains as a cap to many lesions is removed, preferably by using a curette. Thorough and complete removal of the calcified cartilage layer is extremely important based on animal studies we have completed. The integrity of the subchondral plate should be maintained. It is important that the defect is debrided deep enough to remove calcified cartilage layer but not so deep that the subchondral plate is damaged. This prepared lesion, with a stable perpendicular edge of healthy well-attached viable cartilage surrounding the defect provides a pool that helps hold the marrow clot as it forms.

Arthroscopic awls are used to make multiple holes, or “microfractures.” An angled awl, typically 300 or 45°, permits the tip to be perpendicular to the bone as it is advanced. A 90° awl is used for the patella or other soft bone; however, it should only be advanced manually, not with a mallet. Starting at the periphery, microfracture holes are made, ending with holes toward the center of the defect. These are made far enough apart so they do not break into each other, and the subchondral plate between them is protected. Fat droplets from the marrow cavity are seen when the appropriate depth (approximately 2 to 4 mm) has been reached. When completed, the irrigation fluid pump pressure is reduced to observe the release of marrow fat droplets and blood from the microfracture holes. During microfracture, a rough surface has been created in the defect. This surface should not be debrided or shaved further to make it smooth. This rough surface allows for the marrow clot to adhere more easily, yet the integrity of the subchondral plate is maintained for joint surface shape.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning. The methods of the invention also include the use of factor combinations that consist, or consist essentially of the desired factors, for example an implant may consist of a matrix and protein factors consisting of BMP2 surrogate or trispecific proteins.

“Effective amount” generally means an amount which provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

### Conditions for Treatment

The surrogates described herein are useful in methods of treating a cartilage or bone lesion, or injury, in a human or other animal subject, comprising applying to the site a composition comprising an effective dose of the BMP2 surrogate, alone or in combination with a VEGF antagonist, which may be provided in combinations with cements, factors, gels, etc. As referred to herein such lesions include any condition involving skeletal, including cartilaginous, tissue which is inadequate for physiological or cosmetic purposes. Such defects include those that are congenital, the result from disease or trauma, and consequent to surgical or other medical procedures. Such defects include for example, a bone defect resulting from injury, defect brought about during the course of surgery, osteoarthritis, osteoporosis, infection, malignancy, developmental malformation, and bone breakages such as simple, compound, transverse, pathological, avulsion, greenstick and communuted fractures. In some embodiments, a bone defect is a void in the bone that requires filling with a bone progenitor composition.

Inclusion of the factors and/or cells of the invention can be used to facilitate the replacement and filling of cartilage or bone material in and around pre-existing structures. In some embodiments, the cells produce chondrocytes first, followed by deposition of extra cellular matrix and bone formation. The bone grafts can provide an osteoconductive scaffold comprising calcium phosphate ceramics which provide a framework for the implanted progenitor cells and local osteocytes to differentiate into bone forming cells and deposit new bone. The use of calcium phosphate ceramics can provide for a slow degradation of the ceramic, which results in a local source of calcium and phosphate for bone formation. Therefore, new bone can be formed without calcium and phosphate loss from the host bone surrounding the defect site. Calcium phosphate ceramics are chemically compatible to that of the mineral component of bone tissues. Examples of such calcium phosphate ceramics include calcium phosphate compounds and salts, and combinations thereof.

Conditions for regeneration of cartilage include, for example, osteoarthritis (OA), which affects nearly 27 million people in the United States, accounting for 25% of visits to primary care physicians, and half of all NSAID prescriptions. OA is a chronic arthropathy characterized by disruption and potential loss of joint cartilage along with other joint changes, including bone remodeling that may include bone hypertrophy (osteophyte formation), subchondral sclerosis, and formation of subchondral cysts. OA is viewed as failure of the synovial joint. OA results in the degradation of joints, including articular cartilage and subchondral bone, resulting in mechanical abnormalities and impaired joint function. Symptoms may include joint pain, tenderness, stiffness, sometimes an effusion, and impaired joint function. A variety of causes can initiate processes leading to loss of cartilage.

OA may begin with joint damage from trauma to the joint; mechanical injury to the meniscus, articular cartilage, a joint ligament, or another joint structure; defects in cartilage matrix components; and the like. Mechanical stress on joints may underlie the development of OA in many individuals, with many and varied sources of mechanical stress, including misalignments of bones caused by congenital or pathogenic causes; mechanical injury; overweight; loss of strength in muscles supporting joints; and impairment of peripheral nerves, leading to sudden or discoordinated movements that overstress joints.

In synovial joints there are at least two movable bony surfaces that surrounded by the synovial membrane, which secretes synovial fluid, a transparent alkaline viscid fluid which fills the joint cavity, and articular cartilage, which is interposed between the articulating bony surfaces. The earliest gross pathologic finding in osteoarthritis is softening of the articular cartilage in habitually loaded areas of the joint surface. This softening or swelling of the articular cartilage is frequently accompanied by loss of proteoglycans from the cartilage matrix. With progression of osteoarthritis the integrity of the cartilage surface is lost and the articular cartilage thins, with vertical clefts extending into the depth of the cartilage in a process called fibrillation. Joint motion may cause fibrillated cartilage to shed segments that expose the bone underneath (subchondral bone). The subchondral bone is remodeled in OA, including the development of subchondral sclerosis, development of subchondral cysts, and the formation of ectopic bone termed osteophytes. Subchondral cysts also develop which may be filled with synovial fluid. At the joint margins osteophytes (bone spurs) form. The remodeling of subchondral bone increases the mechanical strain and stresses on both the overlying articular cartilage and subchondral bone, leading to further damage of both the cartilage and subchondral bone.

The tissue damage stimulates chondrocytes to attempt repair, which increases production of proteoglycans and collagen. However, efforts at repair also stimulate the enzymes that degrade cartilage, as well as inflammatory cytokines, which are normally present in small amounts. Inflammatory mediators trigger an inflammatory cycle that further stimulates the chondrocytes and synovial lining cells, eventually breaking down the cartilage. Chondrocytes undergo programmed cell death (apoptosis).

OA should be suspected in patients with gradual onset of symptoms and signs, particularly in older adults, usually beginning with one or a few joints. Pain can be the earliest symptom, sometimes described as a deep ache. Pain is usually worsened by weight bearing and relieved by rest but can eventually become constant. Stiffness follows awakening or inactivity. If OA is suspected, plain x-rays should be taken of the most symptomatic joints. X-rays generally reveal marginal osteophytes, narrowing of the joint space, increased density of the subchondral bone, subchondral cyst formation, bony remodeling, and joint effusions. Standing x-rays of knees are more sensitive in detecting joint space narrowing. Magnetic resonance imaging (MRI) can be used to detect cartilage degeneration, and several MRI-based based scoring systems have been developed to characterize the severity of OA.

OA commonly affects the hands, feet, spine, and the large weight bearing joints, such as the hips and knees, although in theory, any joint in the body can be affected. As OA progresses, the affected joints appear larger, are stiff and painful, and usually feel better with gentle use but worse with excessive or prolonged use. Treatment generally involves a combination of exercise, lifestyle modification, and analgesics. If pain becomes debilitating, joint replacement surgery may be used to improve the quality of life.

Among the agents that have demonstrated partial efficacy in control of pain associated with OA are analgesics such as acetaminophen and anti-inflammatories including non-steroidal anti-inflammatory agents (NSAIDs), opiates, intraarticular corticosteroids, and hyaluronic acid derivatives injected into the joint. These agents have not been demonstrated to prevent cartilage loss or slow the loss of joint function.

There are several inflammatory rheumatic diseases that lead to arthritis and can severely damage cartilage tissue. These include rheumatoid arthritis, juvenile idiopathic arthritis, gout, systemic lupus erythematosus, and seronegative spondyloarthropathies.

Rheumatoid arthritis (RA), an autoimmune disease that affects 0.5 to 1% of people worldwide, is characterized by chronic systemic inflammation and arthritis in multiple synovial joints. Left untreated, the disease leads to progressive destruction of cartilage and synovial joints. Serum markers for RA include rheumatoid factor (RF) and anticitrullinated peptide antibody (ACPA). Both adaptive and innate immune systems play a role in the progression of the disease and the major cytokines involved are TNF-α, IL-6, IL-1β and IL-17A. Synthetic disease modifying anti-rheumatic drugs (DMARDs) include methotrexate (which is usually the first line of treatment for RA), leflunomide, hydroxychloroquine and sulfasalazin]. Biologic drugs against TNF-α have constituted a major breakthrough for RA treatment, and constitute the first widespread and major success of the biologic DMARD era; they are effective in halting the progression of the disease in almost 65% of RA patients. Additional therapeutics, when TNF blockers are not found to be effective, include monoclonal antibodies against CD20 in B cells, IL-1 receptor antagonist and monoclonal antibodies against the IL-6 receptor.

Gout is the most common form of inflammatory arthritis, affecting approximately 8.3 million people, or 4% of the population in the United States. It is caused by the deposition of uric acid crystals in the joint and is managed using non-steroidal anti-inflammatory drugs (NSAIDs), colchicine and lifestyle changes to reduce uric acid levels.

Juvenile idiopathic arthritis (JIA) refers to any form of arthritis of unknown cause that occurs in a person younger than 16 years of age. JIA is the most common form of arthritis in children and its prevalence is estimated to range from 16 to 150 per 100,000 in the developed world. In the United States, juvenile arthritis and other rheumatological conditions affect approximately 1 in 250 children. There are six subtypes of JIA: systemic, oligoarticular, polyarticular (which may be positive or negative for rheumatoid factor), juvenile psoriatic, enthesis-related, and undifferentiated arthritis.

Systemic lupus erythematosus is an auto-immune disease characterized by the presence of circulating anti-nuclear antibodies (ANA). Approximately 20% of cases occur in childhood and the childhood onset form of the disease has a worse prognosis compared to the adult onset form. SLE can affect multiple organs in the body including the kidneys, skin, lungs, heart, joints and brain. Clinically, there may be a broad range of signs and symptom. A majority of patients experience arthropathy, and 3 to 6% of patients have Jaccoud's arthropathy which leads to severe deformation of the fingers.

Seronegative spondyloarthropathies, which comprise of ankylosing spondylitis, psoriatic arthritis, reactive arthritis, arthritis associated with inflammatory bowel disease, and a subtype of juvenile idiopathic arthritis. These conditions are characterized by an absence of rheumatoid factor, a strong correlation with carrying the HLA-B27 gene and clinical features that include inflammatory back pain, inflammation of insertion sites of tendons and ligaments, peripheral arthritis and eye inflammation. The overall prevalence of spondyloarthritides in North America is approximately 1% and the most common form of spondyloarthritis is ankylosing spondylitis. The prevalence of psoriatic arthritis in the United States is 0.1% and it is seen in 10 to 40% of people with psoriasis. In a majority of patients, the arthritis develops after psoriasis develops. In addition to arthritis and skin lesions, these patients may also experience eye inflammation, tendinitis and pitting of the fingernails. Importantly, anti-TNF biologics have been found to be very effective in treatment of ankylosing spondylitis as well as psoriatic arthritis.

The BMP2 surrogates also find use in bone generation, e.g. after an injury or trauma resulting in damage. When BMP2 surrogates are used for bone generation, a secondary active agent may also be co-administered. In some embodiments, the secondary active agent may be covalently linked to a BMP2 surrogate of the present disclosure through a cleavable or non-cleavable linker which may be co-administered with a VEGF protein. Secondary active agents that find use in the present disclosure include, without limitation, a WNT activator, a Hedgehog activator, a TGF-β pathway inhibitor, a SIRT1 activator, a ferroptosis inhibitor, a calcineurin inhibitor, a BMP4 agonist, a BMP pathway inhibitor, a VEGF agonist. WNT activators that find use in the present disclosure include, without limitation, CHIR99021, SB-216763, BIO(6-bromoindirubin-3′-oxime), LY2090314, etc. Hedgehog activators that find use in the present disclosure include, without limitation, SAG 21k, SAG, 20(S)-Hydroxycholesterol, etc. TGF-β pathway inhibitors that find use in the present disclosure include, without limitation, A 83-01, SB 431542, etc. Hedgehog activators that find use in the present disclosure include, without limitation, SRT305, SRT1720, resveratrol, etc. Ferroptosis inhibitors that find use in the present disclosure include, without limitation, deferoxamine mesylate, UAMC-3203, etc. Calcineurin inhibitors that find use in the present disclosure include, without limitation, FK506/Tacrolimus, Ciclosporin, etc. BMP4 agonist that find use in the present disclosure include, without limitation, SB 4/Eticovo, etc. BMP pathway activators that find use in the present disclosure include, without limitation, dorsomorphin, SU 6656, etc.

### Methods of Treatment

The present invention provides methods of treating a cartilage lesion, or injury, in a human or other animal subject, comprising applying to the site a composition factors of the invention, which may be provided in combinations with cements, gels, hydrogels, etc. As referred to herein such lesions include any condition involving cartilaginous tissue which is inadequate for physiological or cosmetic purposes. Such defects include those that are congenital, the result from disease or trauma, and consequent to surgical or other medical procedures. Such defects include for example, defect brought about during the course of surgery, osteoarthritis, osteoporosis, infection, malignancy, developmental malformation, etc.

An individual in need of cartilage regeneration can be treated with the methods described herein. An individual for treatment may have osteoarthritis. Various sites for articular cartilage regeneration can be treated, including without limitation knee joint, elbow joint, joints in the phalanges and phalanxes, shoulder joints, hip joints, wrist joints, ankle joints, etc. The individual may be an adult, e.g. past adolescence, and may be an aged adult, e.g. a human over 55 years of age, over 65 years of age, over 70 years of age, etc.

Methods of treatment may comprise inducing an acute local injury. Typically the acute local injury is surgically performed through a microfracture process as described herein, and may be performed with awl, drill, etc.

In some embodiments an individual that presents with an acute local injury, e.g. from accidents, sports injuries, etc., may be beneficially treated with the provision of growth factors in order to reduce development of fibrocartilage and to enhance regeneration of articular cartilage.

At the time of microfracture, or shortly after local acute injury, a drug delivery device is implanted or otherwise positioned to provide an effective dose of BMP2 surrogate or trispecific proteins that serve as both a BMP2 activator and a VEGF inhibitor. The factors may be provided once or multiple times in the course of treatment. For example, an implant comprising factors may be provided to an individual, and additional factors and/or cells provided during the course of treatment.

While in many cases the endogenous SSC are sufficient for regeneration, optionally exogenous cells are provided at the site of local acute injury. The cells may be SSC. The cells may be autologous or allogeneic. The cells may be provided concomitant with the provision of BMP2 surrogate or trispecific proteins, e.g. simultaneously, shortly before, shortly, after, etc. and may be in a single implant with the growth factors, as a separate implant or injection, etc.

### BMP2 Surrogate Proteins

In one respect, this application is directed to multivalent ISV proteins that act as a BMP2 surrogate to activate BMP2 based signaling; optionally in combination with a vascular endothelial growth factor (VEGF) antagonist. The first ISV domain of the surrogate binds to a BMPR1a protein. The second ISV domain of the surrogate binds to a BMPR2 protein. The optional third ISV domain of the surrogate binds to a VEGFA protein. When the surrogate binds to the BMPR1 and BMPR2 proteins, a conformation change occurs, and transphosphorylation events follow thereby activating the BMP2 signaling pathway. The optional third ISV binds to VEGF whereby binding to VEGF antagonizes VEGF function. Collectively, BMP2 pathway signal activation and VEGF antagonism promotes cartilage regeneration.

The ISV domains may be generated using any suitable method. Suitable methods for the generation and screening of ISVs include without limitation, immunization of dromedaries, immunization of camels, immunization of alpacas, immunization of sharks, yeast surface display, etc. Yeast surface display has been successfully used to generate specific ISVs as shown in McMahon et al. (2018) Nature Structural Molecular Biology 25(3): 289-296 which is specifically incorporated herein by reference.

In some embodiments, a first ISV specifically binds to a BMPR1a protein. In some such embodiments, the first ISV specifically binds to the polypeptide of SEQ ID NO:1. Exemplary ISV sequences for this purpose include, for example, those having the amino acid sequence of any of SEQ ID NO:4; SEQ ID NO:8; SEQ ID NO:12; SEQ ID NO:16; SEQ ID NO:20; SEQ ID NO:24; SEQ ID NO:28; or an ISV having the CDR1, CDR2 and CDR3 sequences thereof.

In some embodiments, a second ISV specifically binds to a BMPR2 protein. In some such embodiments, the second ISV specifically binds to the polypeptide of SEQ ID NO:2. Exemplary ISV sequences for this purpose include, for example, those binding to a human BMPR2 protein; and having the amino acid sequence of any of SEQ ID NO:32; SEQ ID NO:36; SEQ ID NO:40; SEQ ID NO:44; SEQ ID NO:48; SEQ ID NO:52; SEQ ID NO:56; SEQ ID NO:60; SEQ ID NO:64; SEQ ID NO:68 or an ISV having the CDR1, CDR2 and CDR3 sequences thereof.

The amino acid sequence of target antigens that find use in the present disclosure include without limitation, the sequence to a BMPR1 a protein, the sequence to a BMPR2 protein, the sequence to a VEGFA protein, etc. An exemplary amino acid sequence to use to generate a first ISV domain directed to human BMPR1 protein may comprise the amino acid sequence: QNLDSMLHGTGMKSDSDQKKSENGVTLAPEDTLPFLKCYCSGHCPDDAINNTCITNGHCFAII EEDDQGETTLASGCMKYEGSDFQCKDSPKAQLRRTIECCRTNLCNQYLQPTLPPVVIGPFFD GSIR (SEQ ID NO: 1). In some embodiments, the target amino acid sequence used to generate the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 1.

An exemplary amino acid sequence to use to generate a second ISV domain directed to human BMPR2 may comprise the amino acid sequence: SQNQERLCAFKDPYQQDLGIGESRISHENGTILCSKGSTCYGLWEKSKGDINLVKQGCWSHIG DPQECHYEECVVTTTPPSIQNGTYRFCCCSTDLCNVNFTENFPPPDTTPLSPPHSFNRDET (SEQ ID NO: 2). In some embodiments, the target amino acid sequence used to generate the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 2.

An exemplary amino acid sequence to use to generate the optional third ISV domain directed to human VEGFA may comprise the amino acid sequence: APMAEGGGQNHHEVVKFMDVYQRSYCHPIETLVDIFQEYPDEIEYIFKPSCVPLMRCGGCCND EGLECVPTEESNITMQIMRIKPHQGQHIGEMSFLQHNKCECRPKKDRARQEKKSVRGKGKGQ KRKRKKSRYKSWSVYVGARCCLMPWSLPGPHPCGPCSERRKHLFVQDPQTCKCSCKNTDS RCKARQLELNERTCRCDKPRR (SEQ ID NO: 3). In some embodiments, the target amino acid sequence used to generate the third ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 3.

The ISV domains are joined through a linker, particularly a polypeptide linker susceptible to proteases found in specifically in monocytes and macrophages, e.g. serine proteases such as chymases, elastases, tryptases, asp-ases, and met-ases. In some embodiments, the protease sensitive linker comprises one or more Val-Cit cleavage sites. In some embodiments, Val-Cit cleavage sites reside between the first and second ISV. In some embodiments the polypeptide linker is from about 8 to about 30 amino acids in length, e.g. from about 10 to about 30, from about 12 to about 30, from about 15 to 30; from about 15-25, from about 15-20 amino acids and is comprised of a poly-(gly-ser) sequence. In some embodiments, the nucleic acid that encodes the sequence of the trispecific protein comprises a TrxA sequence in order to facilitate optimal folding.

In some embodiments, the amino acid sequence of the first ISV domain may comprise the amino acid sequence: QVQLQESGGGLVQAGGSLRLSCAASGNISASYEMGWYRQAPGKERELVAAIDSGASTYYAD SVKGRFTISRDNAKNTVYLQMNSLKPEDTAVYYCAAAGPSYGAPDVIHFYWGQGTQVTVSS (SEQ ID NO: 4). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 4. In some embodiments, the CDR sequences found within the first ISV domain are GNISASYEM (CDR1; SEQ ID NO: 5), ELVAAIDSGASTYY (CDR2; SEQ ID NO: 6) and AAAGPSYGAPDVIHFY (CDR3; SEQ ID NO: 7). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 5, 6, or 7.

In some embodiments, the amino acid sequence of the first ISV domain may comprise the amino acid sequence: QVQLQESGGGLVQAGGSLRLSCAASGTISGRQAMGWYRQAPGKEREFVAGIAYGATTNYAD SVKGRFTISRDNAKNTVYLQMNSLKPEDTAVYYCAAVAVDPEVLWYWGQGTQVTVSS (SEQ ID NO: 8). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 8. In some embodiments, the CDR sequences found within the first ISV domain are GTISGRQAM (CDR1; SEQ ID NO: 9), EFVAGIAYGATTNY (CDR2; SEQ ID NO: 10) and AAVAVDPEVLWY (CDR3; SEQ ID NO: 11). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 9, 10, or 11.

In some embodiments, the amino acid sequence of the first ISV domain may comprise the amino acid sequence: QVQLQESGGGLVQAGGSLRLSCAASGTIFYFSIMGWYRQAPGKEREFVAGIGVGSNTYYADS VKGRFTISRDNAKNTVYLQMNSLKPEDTAVYYCAAFDSISGELQYWGQGTQVTVSS (SEQ ID NO: 12). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 12. In some embodiments, the CDR sequences found within the first ISV domain are GTIFYFSIM (CDR1; SEQ ID NO: 13), EFVAGIGVGSNTYY (CDR2; SEQ ID NO: 14) and AAFDSISGELQY (CDR3; SEQ ID NO: 15). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 13, 14, or 15.

In some embodiments, the amino acid sequence of the first ISV domain may comprise the amino acid sequence: QVQLQESGGGLVQAGGSLRLSCAASGSIFYRGVMGWYRQAPGKERELVAAINDGATTYYAD SVKGRFTISRDNAKNTVYLQMNSLKPEDTAVYYCAVWHRRTGVLLYWGQGTQVTVSS (SEQ ID NO: 16). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 16. In some embodiments, the CDR sequences found within the first ISV domain are GSIFYRGVM (CDR1; SEQ ID NO: 17), ELVAAINDGATTYY (CDR2; SEQ ID NO: 18) and AVWHRRTGVLLY (CDR3; SEQ ID NO: 19). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 17, 18, or 19.

In some embodiments, the amino acid sequence of the first ISV domain may comprise the amino acid sequence: QVQLQESGGGLVQAGGSLRLSCAASGTISVRDPMGWYRQAPGKEREFVAGIGQGSTTNYAD SVKGRFTISRDNAKNTVYLQMNSLKPEDTAVYYCAVLYWRQDWHLYWGQGTQVTVSS (SEQ ID NO: 20). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 20. In some embodiments, the CDR sequences found within the first ISV domain are GTISVRDPM (CDR1; SEQ ID NO: 21), EFVAGIGQGSTTNY (CDR2; SEQ ID NO: 22) and AVLYWRQDWHLY (CDR3; SEQ ID NO: 23). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 21, 22, or 23.

In some embodiments, the amino acid sequence of the first ISV domain may comprise the amino acid sequence: QVQLQESGGGLVQAGGSLRLSCAASGSIFGGDAMGWYRQAPGKERELVAGINAGTSTYYAD SVKGRFTISRDNAKNTVYLQMNSLKPEDTAVYYCAVAGYFPYVHTYWGQGTQVTVSS (SEQ ID NO: 24). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 24. In some embodiments, the CDR sequences found within the first ISV domain are GSIFGGDAM (CDR1; SEQ ID NO: 25), ELVAGINAGTSTYY (CDR2; SEQ ID NO: 26) and AVAGYFPYVHTY (CDR3; SEQ ID NO: 27). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 25, 26, or 27.

In some embodiments, the amino acid sequence of the first ISV domain may comprise the amino acid sequence: QVQLQESGGGLVQAGGSLRLSCAASGTISDGPVMGWYRQAPGKEREFVAGIGYGTNTNYAD SVKGRFTISRDNAKNTVYLQMNSLKPEDTAVYYCAVIVPYNSDRSLYLWYWGQGTQVTVSS (SEQ ID NO: 28). In some embodiments, the amino acid sequence of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 28. In some embodiments, the CDR sequences found within the first ISV domain are GTISDGPVM (CDR1; SEQ ID NO: 29), EFVAGIGYGTNTNY (CDR2; SEQ ID NO: 30) and AVIVPYNSDRSLYLWY (CDR3; SEQ ID NO: 31). In some embodiments, the amino acid sequence of the CDR sequences of the first ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 29, 30, or 31.

In some embodiments, the amino acid sequence of the second ISV domain may comprise the amino acid sequence: QVQLQESGGGLVQAGGSLRLSCAASGNISYYRHMGWYRQAPGKERELVASIGDGGNTNYAD SVKGRFTISRDNAKNTVYLQMNSLKPEDTAVYYCAVVHNTYLLYDPYVWDYLLLYWGQGTQV TVSS (SEQ ID NO: 32). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 32. In some embodiments, the CDR sequences found within the second ISV domain are GTISRQQTM (CDR1; SEQ ID NO: 33), EFVASIAQGGNTYY (CDR2; SEQ ID NO: 34) and AVYERSDTEYYLRY (CDR3; SEQ ID NO: 35). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 33, 34, or 35.

In some embodiments, the amino acid sequence of the second ISV domain may comprise the amino acid sequence: QVQLQESGGGLVQAGGSLRLSCAASGTIFWTASMGWYRQAPGKERELVAGITAGATTYYAD SVKGRFTISRDNAKNTVYLQMNSLKPEDTAVYYCAARTPDRTGIAKWHDYWGQGTQVTVSS (SEQ ID NO: 36). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 36. In some embodiments, the CDR sequences found within the second ISV domain are GTIFWTASM (CDR1; SEQ ID NO: 37), ELVAGITAGATTYY (CDR2; SEQ ID NO: 38) and AARTPDRTGIAKWHDY (CDR3; SEQ ID NO: 39). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 37, 38, or 39.

In some embodiments, the amino acid sequence of the second ISV domain may comprise the amino acid sequence: QVQLQESGGGLVQAGGSLRLSCAASGTIFIPLGMGWYRQAPGKERELVAGITYGGITYYADSV KGRFTISRDNAKNTVYLQMNSLKPEDTAVYYCAARYVVYETHDYWGQGTQVTVSS (SEQ ID NO: 40). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 40. In some embodiments, the CDR sequences found within the second ISV domain are GTIFIPLGM (CDR1; SEQ ID NO: 41), ELVAGITYGGITYY (CDR2; SEQ ID NO: 42) and AARYVVYETHDY (CDR3; SEQ ID NO: 43). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 41, 42, or 43.

In some embodiments, the amino acid sequence of the second ISV domain may comprise the amino acid sequence: QVQLQESGGGLVQAGGSLRLSCAASGTISPRIMGWYRQAPGKEREFVASINDGASTNYADSV KGRFTISRDNAKNTVYLQMNSLKPEDTAVYYCAAAVWYDGWLVYWGQGTQVTVSS (SEQ ID NO: 44). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 44. In some embodiments, the CDR sequences found within the second ISV domain are GTISPRIM (CDR1; SEQ ID NO: 45), EFVASINDGASTNY (CDR2; SEQ ID NO: 46) and AAAVWYDGWLVY (CDR3; SEQ ID NO: 47). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 45, 46, or 47.

In some embodiments, the amino acid sequence of the second ISV domain may comprise the amino acid sequence: QVQLQESGGGLVQAGGSLRLSCAASGNIFRAVNMGWYRQAPGKERELVAAITTGANTYYADS VKGRFTISRDNAKNTVYLQMNSLKPEDTAVYYCAARWYPAPGFNHYYDYWGQGTQVTVSS (SEQ ID NO: 48). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 48. In some embodiments, the CDR sequences found within the second ISV domain are GNIFRAVNM (CDR1; SEQ ID NO: 49), ELVAAITTGANTYY (CDR2; SEQ ID NO: 50) and AARWYPAPGFNHYYDY (CDR3; SEQ ID NO: 51). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 49, 50, or 51.

In some embodiments, the amino acid sequence of the second ISV domain may comprise the amino acid sequence: QVQLQESGGGLVQAGGSLRLSCAASGSISSATVMGWYRQAPGKEREFVAAITAGGTTNYADS VKGRFTISRDNAKNTVYLQMNSLKPEDTAVYYCAVVVFTSQDRIYYYRYWGQGTQVTVSS (SEQ ID NO: 52). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 52. In some embodiments, the CDR sequences found within the second ISV domain are GSISSATVM (CDR1; SEQ ID NO: 53), EFVAAITAGGTTNY (CDR2; SEQ ID NO: 54) and AVVVFTSQDRIYYYRY (CDR3; SEQ ID NO: 55). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 53, 54, or 55.

In some embodiments, the amino acid sequence of the second ISV domain may comprise the amino acid sequence: QVQLQESGGGLVQAGGSLRLSCAASGSIFPAQVMGWYRQAPGKERELVAGINYGGSTYYAD SVKGRFTISRDNAKNTVYLQMNSLKPEDTAVYYCAVFQWDDNDGRYYLGYWGQGTQVTVSS (SEQ ID NO: 56). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 56. In some embodiments, the CDR sequences found within the second ISV domain are GSIFPAQVM (CDR1; SEQ ID NO: 57), ELVAGINYGGSTYY (CDR2; SEQ ID NO: 58) and AVFQWDDNDGRYYLGY (CDR3; SEQ ID NO: 59). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 57, 58, or 59.

In some embodiments, the amino acid sequence of the second ISV domain may comprise the amino acid sequence: QVQLQESGGGLVQAGGSLRLSCAASGTIFHGSTMGWYRQAPGKERELVAGIASGSSTYYAD SVKGRFTISRDNAKNTVYLQMNSLKPEDTAVYYCAVAQQQHGTDRSWFIYWGQGTQVTVSS (SEQ ID NO: 60). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 60. In some embodiments, the CDR sequences found within the second ISV domain are GTIFHGSTM (CDR1; SEQ ID NO: 61), ELVAGIASGSSTYY (CDR2; SEQ ID NO: 62) and AVAQQQHGTDRSWFIY (CDR3; SEQ ID NO: 63). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 61, 62, or 63.

In some embodiments, the amino acid sequence of the second ISV domain may comprise the amino acid sequence: QVQLQESGGGLVQAGGSLRLSCAASGTISIYGPMGWYRQAPGKERELVAGISLGGSTNYADS VKGRFTISRDNAKNTVYLQMNSLKPEDTAVYYCAAASIAKSDYPYWHLYWGQGTQVTVSS (SEQ ID NO: 64). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 64. In some embodiments, the CDR sequences found within the second ISV domain are GTISIYGPM (CDR1; SEQ ID NO: 65), ELVAGISLGGSTNY (CDR2; SEQ ID NO: 66) and AAASIAKSDYPYWHLY (CDR3; SEQ ID NO: 67). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 65, 66, or 67.

In some embodiments, the amino acid sequence of the second ISV domain may comprise the amino acid sequence: QVQLQESGGGLVQAGGSLRLSCAASGTISARKRMGWYRQAPGKEREFVATIDYGTTTNYADS VKGRFTISRDNAKNTVYLQMNSLKPEDTAVYYCAVIDYVPIDYYQYAAGWHFYWGQGTQVTV SS (SEQ ID NO: 68). In some embodiments, the amino acid sequence of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 68. In some embodiments, the CDR sequences found within the second ISV domain are GNISPTYLM (CDR1; SEQ ID NO: 69), EFVAGIAHGASTNY (CDR2; SEQ ID NO: 70) and AVNPYALDVLVY (CDR3; SEQ ID NO: 71). In some embodiments, the amino acid sequence of the CDR sequences of the second ISV domain has at least 60%, at least 65%, at least 70%, at least 75%, at least 80%, at least 85%, at least 90%, at least 95%, at least 98%, at least 99%, or 100%, amino acid sequence identity with that of SEQ ID NO: 69, 70, or 71.

### Kits

Also provided are kits for use in the methods. The agents of a kit can be present in the same or separate containers. The agents may also be present in the same container. In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

### Example 1

### Treatment of Cartilage Damage

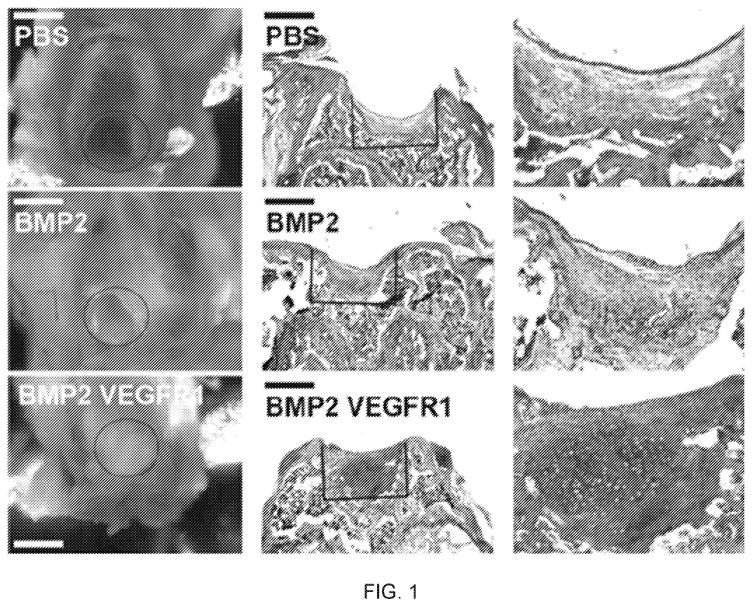

The OA knee joints were microfractured, and then previously validated PEG hydrogel constructs were delivered, treated with combinations of BMP2 and sVEGFR1, to the (microfracture) MF site. The recipient mice was allowed to heal for 4 weeks before re-isolating joints for histological analysis. PEG hydrogels were found loaded with control PBS led to a fibrotic-tissue defect with minimal cartilage or bone regeneration, whereas PEG hydrogels loaded with BMP2 promoted substantial bone formation at the MF site (FIG. 1). In contrast, BMP2+sVEGFR1 stimulated robust formation of cartilage at the MF site that stained intensely blue for proteoglycan by Movat's Pentachrome and contained numerous morphologically distinct chondrocytes (FIG. 1).

### Mouse Microfracture Model

Nine-week-old, skeletally mature, sex-matched C57BL/6 or β Actin-CreERT/Rainbow mice are used to examine the effect of MF on the resident mSSC population in combination with delivery of a BMP2 surrogate disclosed herein. Under general anesthesia (isoflurane), after assessing the pain response of the mice by using a toe pinch, a 5-mm incision is made medial to knee, reflecting the patella laterally, applying a drop of sterile 0.9% sodium chloride and flexing the knee to expose the femoral condyles. Microfracturing is performed on the articular surface of the left femur (0.1-mm diameter to access underling subchondral bone). The right femur undergoes incisions to expose patella but without MF and acts as a control (sham). The patella is repositioned. The incision is closed with 6-0 prolene suture.

### Mouse Osteoarthritis Model

Mice are anesthetized with general anesthetic with inhaled isoflurane and a preoperative dose of 0.5 ml per kg (body weight) of buprenorphine. Routine preparation and surgical draping of each animal around the surgical site includes: shaving the leg, a thorough cleaning with 70% alcohol/betadine and draping with sterile gauze. Surgical instruments are sterilized with heat. A 5-mm incision is made medial to knee. The patella is reflected laterally. A drop of sterile 0.9% sodium chloride is applied. The knee is flexed to expose the femoral chondyles. A small vertical incision is made to the meniscotibial ligament of the medial meniscus and delivery of a BMP2 surrogate disclosed herein is made. The patella was repositioned. The incision is closed with 6-0 prolene.

### Hydrogel Fabrication

Eight-arm PEG monomers with end groups of norbornene (molecular weight, 10 kDa) or mercaptoacetic ester (molecular weight, 10 kDa) were dissolved in PBS at a concentration of 20% (wt/vol). Photoinitiator lithium phenyl-2,4,6-trimethylbenzoylphosphinate was then added to each solution to make a concentration of 0.05% (wt/vol). The two polymer solutions were mixed at a 1:1 volume ratio to obtain a hydrogel precursor solution. Recombinant BMP2 and sVEGFR1 (R&D Systems) were added to precursor solution to reach predetermined loading amount. Solutions were sandwiched between 2 slides with a thickness of 400 μm and exposed to ultraviolet light (365 nm, 4 mW per cm2) for 5 min. The hydrogels were made with volume of 1 μl or 10 μl. The growth factor loading per each 6 μl hydrogel included: 3 μg BMP2±25 μg sVEGFR1. The hydrogels were made without growth factors and served as controls.

### ISV Screening and Production

For ISV screening, flag tagged antigen (corresponding to SEQ ID NO: 1-3) was incubated with induced yeast library, then stained with AF647 conjugated anti-flag antibody and sorted for AF647 positive yeast by magnetic-activated cell sorting (MACS) or fluorescence-activated cell sorting (FACS). After several rounds of selection, antigen binding yeast were enriched to above 20% of the total population. Antigen binding clones were sequenced to identify nanobody sequences that bound antigen. Nanobodies were expressed in E. coli and purified using Ni-NTA beads. For trispecific ISVs, NANOBODY® against a BMPR1a, NANOBODY® against a BMPR2 protein and NANOBODY® against VEGFA were joined by GS linker comprising a Val-Cit cleavage site between the first and the second ISV but not the third.

### Example 2

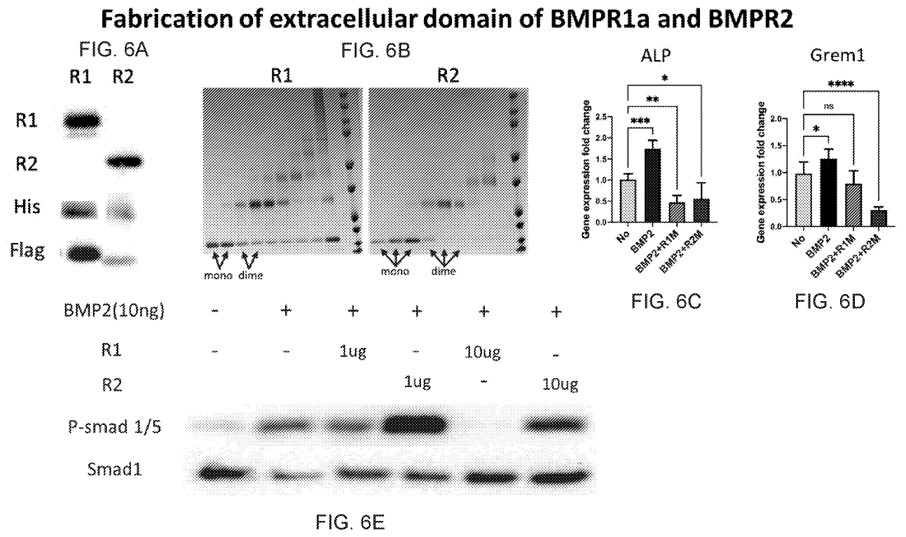

As shown in FIG. 6, the extracellular domain (ECD) of BMPR1a and BMPR2 were synthesized with HIS and FLAG tag using E. coli with pET 26 vector. ECD of BMPRs were purified with nickel ion chromatography using His-tag, and expression confirmed by western blot (FIG. 6a). Size chromatography was used to isolate the monomer form of BMPR1a and BMPR2 (FIG. 6b). To examine the functional activity of BMPR1a, SSCs were treated with ECD of BMPR1a or BMPR2 together with BMP2 and the downstream signaling such as ALP and Grem1 examined to test if fabricated ECD of BMPRs block the activity of BMP2. If ECD of BMPRs are functional, it should block the downstream signaling. FIGS. 6c and d shows the qPCR results, showing that fabricated ECD of BMPRs block the ALP and Grem1 activity, indicating the successful fabrication of BMPR1a and BMPR2. To further confirm the activity of ECD of BMPRs, SSCs were treated with BMP2 (10 ng ng/ml) together with indicated amount of ECD of BMPRs for 30 minutes and the p-smad1/5 activity examined. P-smad1/5 signaling has been abrogated by addition of BMPR1a. Interestingly, p-smad1/5 activity was increased by addition of ECD of BMPR2 (1 ug/ml) and reduced by 10 ug/ml of BMPR2. This is because ECD of BMPR2 acts as a support to induce p-smad signaling up to a certain concentration, but it starts to block the BMP2 activity with very high concentration (FIG. 1e).

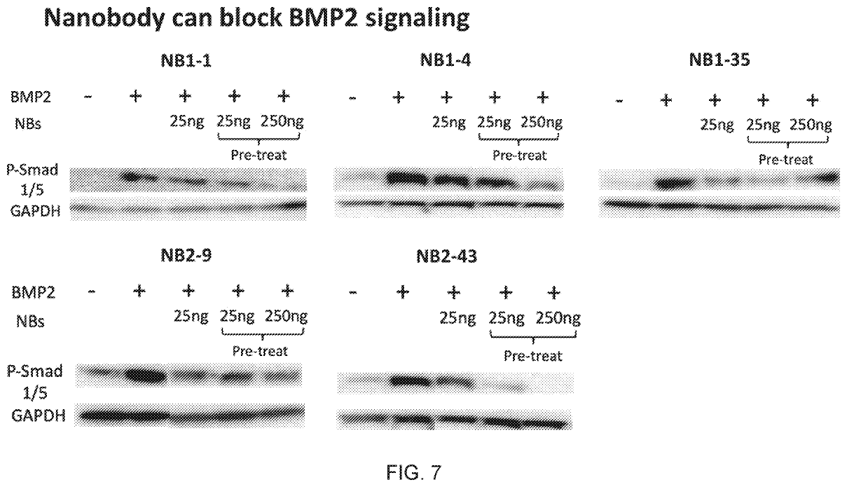

Next, the ECD of the two BMPRs was used as antigens to select candidate nanobodies, designated as NB1 and NB2 respectively, against BMPR1a and BMPR2, with a yeast library. After two rounds of magnetic activated cell sorting and four rounds of fluorescent activated cell sorting, 40 candidate NBs were obtained for each receptor. The functional activity of the NBs was examined by culturing SSCs and treating them with an NB1 or NB2 protein (25 ng/ml) together with BMP2 (2.5 ng/ml). If the NBs are functional, they should competitively binds to BMPRs and block the BMP2 signaling. To enhance the blocking effect of NBs, 5 minutes pre-treatment was also performed with NB1 or NB2 (25 ng/ml or 250 ng/ml). The representative results for NB1a and NB2 are shown in FIG. 7. The treatment with NBs significantly reduced the phosphorylation of smad1/5.

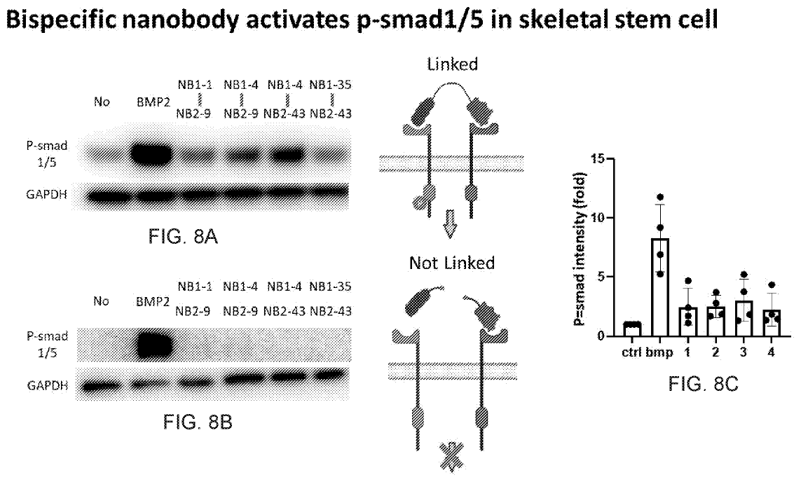

The NB1 and NB2 proteins were linked by a flexible GS linker (8 aa) to fabricate a surrogate BMP2. FIG. 8a shows the p-smad1/5 signaling after 30 minutes treatment of surrogate BMP2s. Four combinations are listed in the figure. Of these, (NB1-4)-(NB2-43) shows the best signaling activation (3 folds higher activation than control). When NBs were administered separately, without linker conjugation, no p-smad1/5 activation was observed, indicating that NB1 and NB2 instead block the receptors (FIG. 8b)

Exemplary bispecific proteins have sequences as follows:

(NB1-1)-(NB2-9),
SEQ ID NO: 72
QVQLQESGGGLVQAGGSLRLSCAASGNISASYEMGWYRQAPGKERELVAA
IDSGASTYYADSVKGRFTISRDNAKNTVYLQMNSLKPEDTAVYYCAAAGP
SYGAPDVIHFYWGQGTQVTVSSGGGGSGGGQVQLQESGGGLVQAGGSLRL
SCAASGTISIYGPMGWYRQAPGKERELVAGISLGGSTNYADSVKGRFTIS
RDNAKNTVYLQMNSLKPEDTAVYYCAAASIAKSDYPYWHLYWGQGTQVTV
SS
(NB1-4)-(NB2-9)
SEQ ID NO: 73
QVQLQESGGGLVQAGGSLRLSCAASGTIFYFSIMGWYRQAPGKEREFVAG
IGVGSNTYYADSVKGRFTISRDNAKNTVYLQMNSLKPEDTAVYYCAAFDS
ISGELQYWGQGTQVTVSSGGGGSGGGQVQLQESGGGLVQAGGSLRLSCAA
SGTISIYGPMGWYRQAPGKERELVAGISLGGSTNYADSVKGRFTISRDNA
KNTVYLQMNSLKPEDTAVYYCAAASIAKSDYPYWHLYWGQGTQVTVSS
(NB1-4)-(NB2-43)
SEQ ID NO: 74
QVQLQESGGGLVQAGGSLRLSCAASGTIFYFSIMGWYRQAPGKEREFVAG
IGVGSNTYYADSVKGRFTISRDNAKNTVYLQMNSLKPEDTAVYYCAAFDS
ISGELQYWGQGTQVTVSSGGGGSGGGQVQLQESGGGLVQAGGSLRLSCAA
SGSISSATVMGWYRQAPGKEREFVAAITAGGTTNYADSVKGRFTISRDNA
KNTVYLQMNSLKPEDTAVYYCAVVVFTSQDRIYYYRYWGQGTQVTVSS
(NB1-35)-(NB2-43)
SEQ ID NO: 75
QVQLQESGGGLVQAGGSLRLSCAASGSIFYRGVMGWYRQAPGKERELVAA
INDGATTYYADSVKGRFTISRDNAKNTVYLQMNSLKPEDTAVYYCAVWHR
RTGVLLYWGQGTQVTVSSGGGGSGGGQVQLQESGGGLVQAGGSLRLSCAA
SGSISSATVMGWYRQAPGKEREFVAAITAGGTTNYADSVKGRFTISRDNA
KNTVYLQMNSLKPEDTAVYYCAVVVFTSQDRIYYYRYWGQGTQVTVSS

1. A human BMP2 surrogate protein comprises a first antigen binding region that specifically binds to a first human BMP2 receptor and a second antigen binding region that specifically binds to a second human BMP2 receptor.

2. The human BMP2 surrogate protein of claim 1, wherein the first and the second antigen binding regions are configured as immunoglobulin “single variable domains” (ISV).

3. The human BMP2 surrogate protein of claim 2, wherein the first and second ISV are V_{HH} domains.

4. The human BMP2 surrogate protein of claim 1, wherein the first and the second antigen binding regions are separated by a linker from 8 to 30 amino acids in length; optionally (i) a polypeptide susceptible to proteases selectively expressed in monocytic cells or macrophages, relative to skeletal stem cells, or (ii) comprising one or more valine-citrulline sequences.

5-7. (canceled)

8. The human BMP2 surrogate protein of claim 1, wherein the first antigen binding region comprises a variable heavy homodimer (VHH) domain with the amino acid sequence of any of SEQ ID NO:4; SEQ ID NO:8; SEQ ID NO:12; SEQ ID NO:16; SEQ ID NO:20; SEQ ID NO:24; SEQ ID NO:28 or an ISV having the CDR1, CDR2 and CDR3 sequences of SEQ ID NO:4; SEQ ID NO:8; SEQ ID NO:12; SEQ ID NO:16; SEQ ID NO:20; SEQ ID NO:24; SEQ ID NO:28.

9. The human BMP2 surrogate protein of claim 1, wherein the second ISV comprises a variable heavy homodimer (VHH) domain having the amino acid sequence of any of SEQ ID NO:32; SEQ ID NO:36; SEQ ID NO:40; SEQ ID NO:44; SEQ ID NO:48; SEQ ID NO:52; SEQ ID NO:56; SEQ ID NO:60; SEQ ID NO:64; SEQ ID NO:68; or an ISV having the CDR1, CDR2 and CDR3 sequences of any of SEQ ID NO:32; SEQ ID NO:36; SEQ ID NO:40; SEQ ID NO:44; SEQ ID NO:48; SEQ ID NO:52; SEQ ID NO:56; SEQ ID NO:60; SEQ ID NO:64; SEQ ID NO:68.

10. The human BMP2 surrogate protein of claim 1, wherein the first antigen binding region that specifically binds to a first human BMP2 receptor is selected from NB1-1, NB1-4 and NB1-35.

11. The human BMP2 surrogate protein of claim 1, wherein the second antigen binding region that specifically binds to a first human BMP2 receptor is selected from NB2-9 and NB2-43.

12. The human BMP2 surrogate protein of claim 1, comprising an amino acid sequence selected from SEQ ID NO:72; SEQ ID NO:73; SEQ ID NO:74; and SEQ ID NO:75.

13. The human BMP2 surrogate protein of claim 1, further comprising an antigen binding region that specifically binds and inhibits activity of human VEGF.

14. An implantable drug delivery device comprising an effective dose of a BMP2 surrogate protein according to claim 1.

15. The implantable drug device of claim 14, further comprising an effective dose of a second active agent that enhanced regeneration of bone or cartilage.

16. The implantable drug device of claim 14, wherein the drug delivery device is a non-biodegradable implant optionally a reservoir implant or a monolithic implant, or a biodegradable implant.

17-18. (canceled)

19. The implantable drug device of claim 16, wherein the biodegradable implant is a block polymer implant, optionally comprising poly(caprolactone) (PCL), poly(lactic acid) (PLA) or poly(lactic-co-glycolic acid) (PLGA).

20-21. (canceled)

22. The implantable drug device of claim 16, wherein the biodegradable implant is a hydrogel.

23. A method for regenerating mammalian cartilage, the method comprising:
implanting in an individual a drug device according to claim 16 at a target site where cartilage regeneration is desired, wherein the drug device comprises an antagonist of VEGF.

24. The method of claim 23, wherein the cartilage is articular cartilage.

25. A method for regenerating mammalian bone, the method comprising:
implanting in an individual a drug device according to claim 1 at a target site where bone regeneration is desired, wherein the drug device optionally comprises a second active agent that enhances bone formation.

26. (canceled)

27. The method of claim 23, wherein the drug delivery device is implanted at the targeted site with an effective dose of skeletal stem cells.

28. The method of any of claims 23-27 wherein the targeted site is the site of an acute local injury, optionally a surgically performed microfracture procedure to bone tissue.

29. (canceled)
